(19) United States
(12) Patent Application Publication
(10) Pub. No.: US 2020/0024299 A1
(43) Pub. Date: Jan. 23, 2020

(54) CRYSTALLINE FORMS OF A BILE ACID DERIVATIVE

(71) Applicant: Intercept Pharmaceuticals, Inc., New York, NY (US)

(72) Inventors: Rachel Pineda Strazik, Encinitas, CA (US); Kevin Schaab, Spring Valley, CA (US); Alex Eberlin, Cambridge (GB); Rosa Maria Espinosa, Cambridge (GB)

(21) Appl. No.: 16/337,982

(22) PCT Filed: Sep. 29, 2017
(86) PCT No.: PCT/US17/54227

§ 371 Date: Mar. 29, 2019

Related U.S. Application Data
(60) Provisional application No. 62/402,813, filed on Sep. 30, 2016.

(51) Int. Cl. C07J 31/00; A61K 31/575

(52) U.S. Cl. CPC C07J 31/006; C07B 2200/13; A61K 31/575

(57) ABSTRACT

A crystalline form of a bile acid compound and methods of preparation and use thereof are described.

### BACKGROUND

Farnesoid X Receptor (FXR) is a member of the nuclear receptor family of ligand-activated transcription factors that includes receptors for the steroid, retinoid, and thyroid hormones. FXR is most abundantly expressed in the liver, intestine, kidney, and adrenal gland. FXR binds to DNA as a heterodimer with the 9-cis retinoic acid receptor (RXR). Several naturally-occurring bile acids bind to and activate FXR at physiological concentrations. Bile acids that serve as FXR ligands include chenodeoxycholic acid (CDCA), deoxycholic acid (DCA), lithocholic acid (LCA), and the taurine and glycine conjugates of these bile acids.

Bile acids are cholesterol metabolites that are formed in the liver and secreted into the duodenum of the intestine, where they have important roles in the solubilization and absorption of dietary lipids and vitamins. Most bile acids (˜95%) are subsequently reabsorbed in the ileum and returned to the liver via the enterohepatic circulatory system. The conversion of cholesterol to bile acids in the liver is under feedback regulation: bile acids down-regulate the transcription of cytochrome P450 7a (CYP7a), which encodes the enzyme that catalyzes the rate limiting step in bile acid biosynthesis. There is data to suggest that FXR is involved in the repression of CYP7a expression by bile acids. In the ileum, bile acids induce the expression of the intestinal (ileal) bile acid binding protein (IBABP), a cytoplasmic protein which binds bile acids with high affinity and may be involved in their cellular uptake and trafficking. Two groups have now demonstrated that bile acids mediate their effects on IBABP expression through activation of FXR, which binds to an IR-1 type response element that is conserved in the human, rat, and mouse IBABP gene promoters. Thus FXR is involved in both the stimulation (IBABP) and the repression (CYP7a) of target genes involved in bile acid and cholesterol homeostasis.

Accordingly, new compounds and methods for modulating FXR for the treatment or prevention of an FXR-mediated diseases or disorders are needed. The present application addresses these needs.

### SUMMARY

In one aspect, this application pertains to crystalline forms of Compound 1-Na.

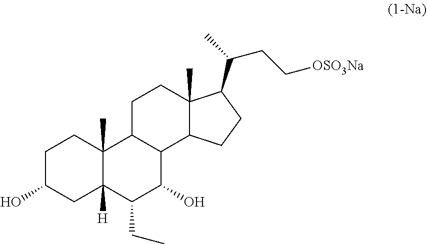

In one embodiment, the crystalline form of Compound 1-Na is Form A, wherein Form A is characterized by having X-ray powder diffraction (XRPD) peaks at approximately 8.5, 15.8, and 16.7° 2θ (theta) using Cu Kα radiation.

In one embodiment, the crystalline form of Compound 1-Na is Form A, wherein Form A is characterized by having an orthorhombic crystal system with the following unit cell parameters: a=approximately 8.7 Å, b=approximately 27.0 Å, and c=approximately 34.8 Å.

In one embodiment, the crystalline form of Compound 1-Na is Form A, wherein Form A is characterized by having an orthorhombic space group P2₁P2₁P2₁.

In one aspect, this application pertains to a pharmaceutical composition comprising a crystalline form of Compound 1-Na (i.e., Form A), and a pharmaceutically acceptable diluent, excipient, or carrier.

In one aspect, this application pertains to a method of treating or preventing an FXR-mediated disease or disorder in a subject in need thereof, comprising administering a therapeutically effective amount of a crystalline form of Compound 1-Na (i.e., Form A) or a pharmaceutical composition comprising a crystalline form of Compound 1-Na (e.g., Form A). In one aspect, this application pertains to a crystalline form of Compound 1-Na (i.e., Form A) or a pharmaceutical composition comprising a crystalline form of Compound 1-Na (i.e., Form A) for treating or preventing an FXR-mediated disease or disorder.

In one aspect, this application pertains to use of a crystalline form of Compound 1-Na (i.e., Form A) or a pharmaceutical composition comprising a crystalline form of Compound 1-Na (i.e., Form A) in the manufacture of a medicament for treating or preventing an FXR-mediated disease or disorder.

In one aspect, this application pertains to a method of modulating FXR in a subject in need thereof, comprising administering a therapeutically effective amount of a crystalline form of Compound 1-Na (i.e., Form A) or a pharmaceutical composition comprising a crystalline form of Compound 1-Na (i.e., Form A).

In one aspect, this application pertains to a crystalline form of Compound 1-Na (i.e., Form A) or a pharmaceutical composition comprising a crystalline form of Compound 1-Na (i.e., Form A) for modulating FXR.

In one aspect, this application pertains to use of a crystalline form of Compound 1-Na (i.e., Form A) or a pharmaceutical composition comprising a crystalline form of Compound 1-Na (i.e., Form A) in the manufacture of a medicament for modulating FXR.

Unless otherwise defined, all technical and scientific terms used herein have the same meaning as commonly understood by one of ordinary skill in the art to which this application belongs. In the specification, the singular forms also include the plural unless the context clearly dictates otherwise. Although methods and materials similar or equivalent to those described herein can be used in the practice or testing of the present application, suitable methods and materials are described below. All publications, patent applications, patents, and other references mentioned herein are incorporated by reference. The references cited herein are not admitted to be prior art. In the case of conflict, the present specification, including definitions, will control. In addition, the materials, methods, and examples are illustrative only and are not intended to be limiting.

Other features and advantages of the application will be apparent from the following detailed description and claims.

### BRIEF DESCRIPTION OF THE FIGURES

FIG. 1 shows XRPD diffractogram of Amorphous Form of Compound 1-Na.

FIG. 2 shows ¹H NMR Spectrum of Amorphous Form of Compound 1-Na.

FIG. 3 shows XRPD diffractogram of the Crystalline Form A of Compound 1-Na formed via suspension.

FIG. 4 shows ¹H NMR Spectrum of the Chrystalline Form A of Compound 1-Na formed via suspension.

FIG. 5 shows DSC and TGA Thermograms of the Crystallinine Form A of Compound 1-Na formed via suspension.

FIG. 6 shows stability of the Crystalline Form A of Compound 1-Na Formed via suspension for 1 month at 25° C./60% RH.

FIG. 7 shows comparison of XRPDs of the Crystalline Form A of Compound 1-Na formed in Examples 3, 5, and 6.

FIG. 8 shows DSC and TGA Thermograms of the Crystalline Form A of Compound 1-Na formed in Example 5.

FIG. 9 shows DSC and TGA Thermograms of the Crystalline Form A of Compound 1-Na formed in Example 6.

FIG. 10 shows ¹H NMR Spectrum of the Crystalline Form A of Compound 1-Na formed in Example 5.

FIG. 11 shows Variable Temperature (VT) XRPD Analysis of the Crystalline Form A of Compound 1-Na formed in Example 5.

FIG. 12 shows DVS Isotherm Plot for the Crystalline Form A of Compound 1-Na formed in Example 5.

FIG. 13 show shows DVS Mass plot for the Crystalline Form A of Compound 1-Na Formed in Example 5.

FIG. 14 shows PLM images of the Crystalline Form A of Compound 1-Na formed in Example 5.

FIG. 15 shows Electronic Microscopic images of the Crystalline Form A of Compound 1-Na formed in Example 5.

FIG. 16 shows ¹H NMR Spectrum of the Crystalline Form A of Compound 1-Na formed in Example 5 after drying at 40° C.

FIG. 17 shows XRPD Analysis of the Crystalline Form A of Compound 1-Na formed in Example 5 after drying at 40° C.

FIG. 18 shows XRPD diffractogram of the Crystalline Form A of Compound 1-Na formed in Example 6 measured from capillary data.

FIG. 19 shows XPRD diffractogram of the Crysyalline Form A of Compound 1-Na.

FIG. 20 shows XRPD diffractogram of the Crystalline Form of Compound 1-OH.

### DETAILED DESCRIPTION

In one aspect, this application pertains to crystalline forms of Compound 1-Na.

In one embodiment, the crystalline form of Compound 1-Na is Form A, wherein Form A is characterized by having XRPD peaks at approximately 8.5, 15.8, and 16.7° 2θ (theta) using Cu Kα radiation.

In one embodiment, the crystalline form of Compound 1-Na is Form A, wherein Form A is characterized by having XRPD peaks at approximately 4.0, 8.5, 15.8, 16.7, 17.8, and 18.2° 2θ (theta) using Cu Kα radiation.

In one embodiment, the crystalline form of Compound 1-Na is Form A, wherein Form A is characterized by having XRPD peaks at approximately 4.0, 6.6, 7.1, 8.5, 11.5, 13.5, 15.8, 16.7, 17.8, and 18.2° 2θ (theta) using Cu Kα radiation.

In one embodiment, the crystalline form of Compound 1-Na is Form A, wherein Form A is characterized by having XRPD peaks at 8.5±0.2° two theta, 15.8±0.2° two theta, and 16.7±0.2° two theta using Cu Kα radiation.

In one embodiment, the crystalline form of Compound 1-Na is Form A, wherein Form A is characterized by having XRPD peaks at 4.0±0.2° two theta, 8.5±0.2° two theta, 15.8±0.2° two theta, 16.7±0.2° two theta, 17.8±0.2° two theta, and 18.2±0.2° two theta using Cu Kα radiation.

In one embodiment, the crystalline form of Compound 1-Na is Form A, wherein Form A is characterized by having XRPD peaks at 4.0±0.2° two theta, 6.6±0.2° two theta, 7.1±0.2° two theta, 8.5±0.2° two theta, 11.5±0.2° two theta, 13.5±0.2° two theta, 15.8±0.2° two theta, 16.7±0.2° two theta, 17.8±0.2° two theta, and 18.2±0.2° two theta using Cu Kα radiation.

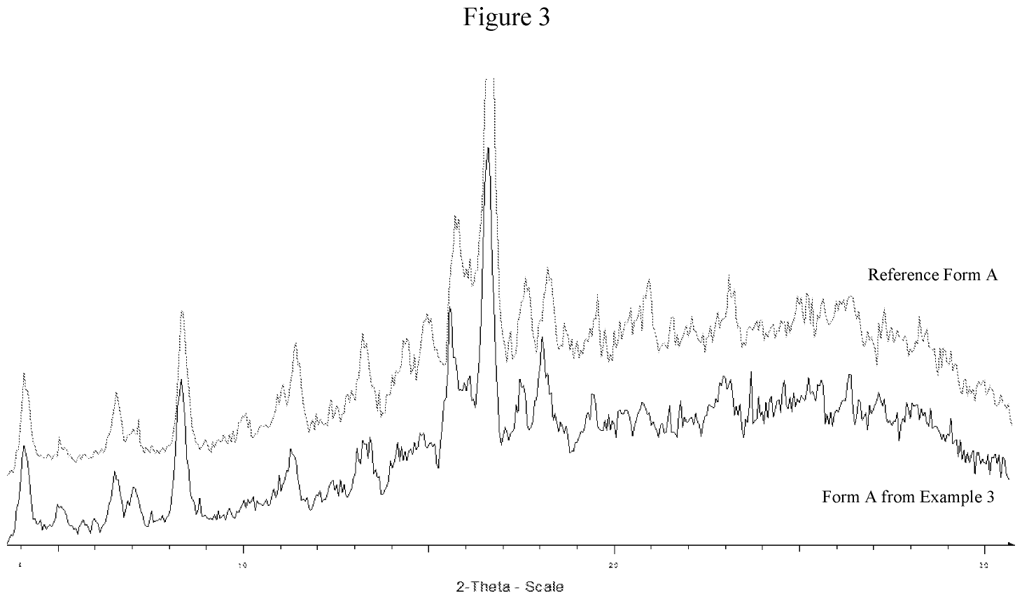

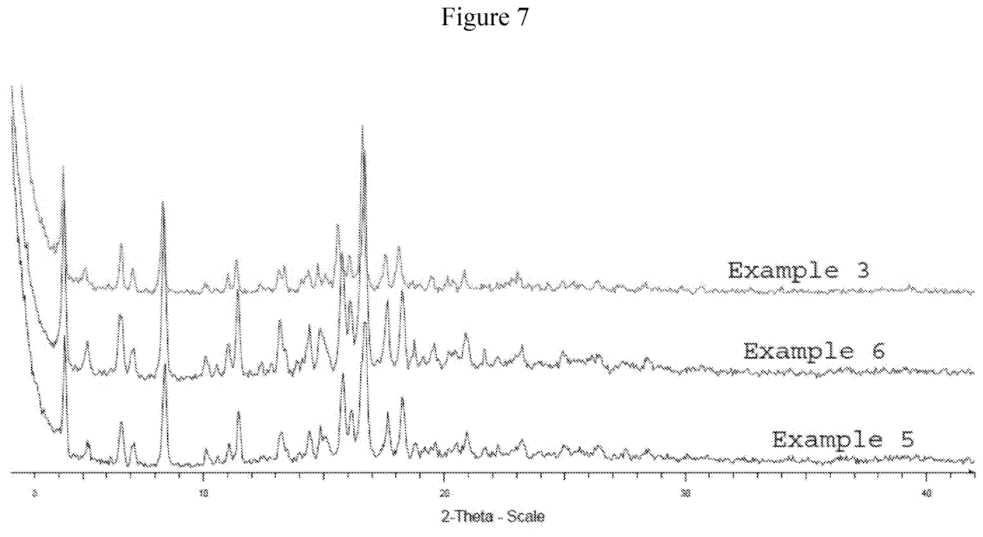

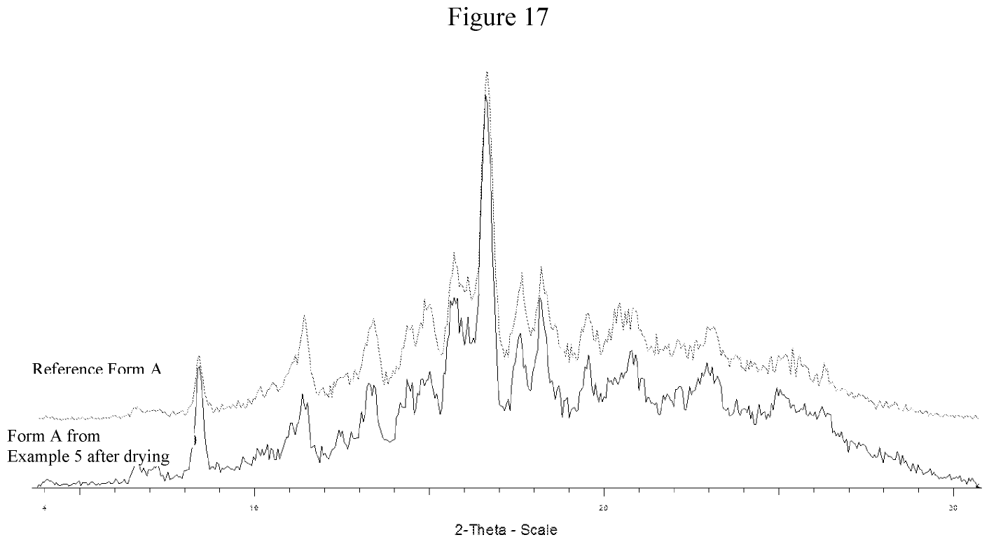

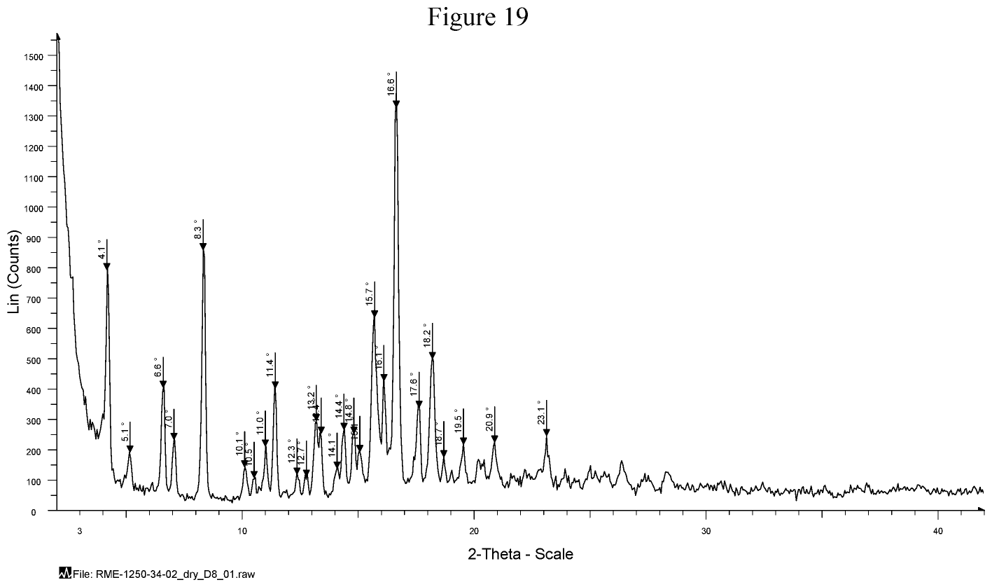

In one embodiment, the crystalline form of Compound 1-Na is Form A, wherein Form A is characterized by having an XRPD pattern substantially similar to that shown in FIG. 3, FIG. 7, FIG. 17 or FIG. 19.

In one embodiment, the crystalline form of Compound 1-Na is Form A, wherein Form A is characterized by having an XRPD pattern substantially similar to that shown in FIG. 7.

In one embodiment, the crystalline form of Compound 1-Na is Form A, wherein Form A is characterized by having an orthorhombic crystal system with the following unit cell parameters: a=approximately 8.7 Å, b=approximately 27.0 Å, and c=approximately 34.8 Å. In one embodiment, the crystalline form of Compound 1-Na is Form A, wherein Form A is characterized by a total volume of the basic unit cell being around 8000-8300 Å³. In one embodiment, the crystalline form of Compound 1-Na is Form A, wherein Form A is characterized by a total volume of the basic unit cell being around 8181.4 Å³.

In one of the embodiments, the crystalline Form A of Compound 1-Na has a higher probability of having an orthorhombic space group P2₁2₁2₁ than the other groups.

In one of the embodiments the crystalline Form A of Compound 1-Na is characterized by having an orthorhombic space group P2₁2₁2₁.

The space group determination can be conducted and assessed via the “Pawley” fitting procedure.

In one embodiment, the crystalline form of Compound 1-Na is Form A, wherein Form A is characterized by a Differential Scanning calorimetry (DSC) having an onset temperature between about 159° C. and about 172° C. In one embodiment, the crystalline form of Compound 1-Na is Form A, wherein Form A is characterized by a DSC having an onset temperature at approximately 165° C. to 169° C. In one embodiment, the crystalline form of Compound 1-Na is Form A, wherein Form A is characterized by a DSC having an onset temperature at approximately 167° C.

In one embodiment, the polymorph of Compound 1-Na is Form A, wherein Form A is characterized by a DSC having an onset temperature between about 27° C. and about 30° C. In one embodiment, the crystalline form of Compound 1-Na is Form A, wherein Form A is characterized by a DSC having an onset temperature at approximately 29° C.

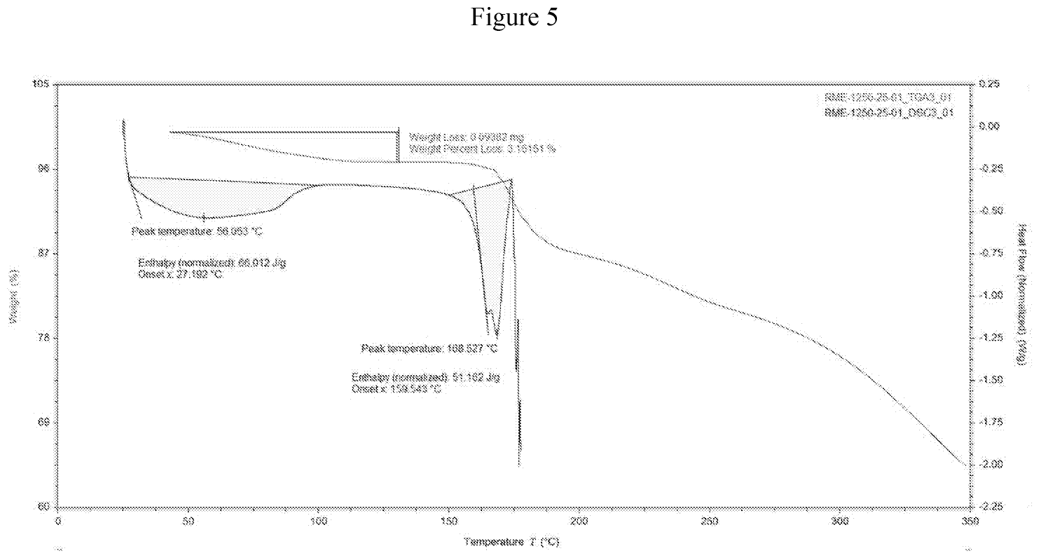

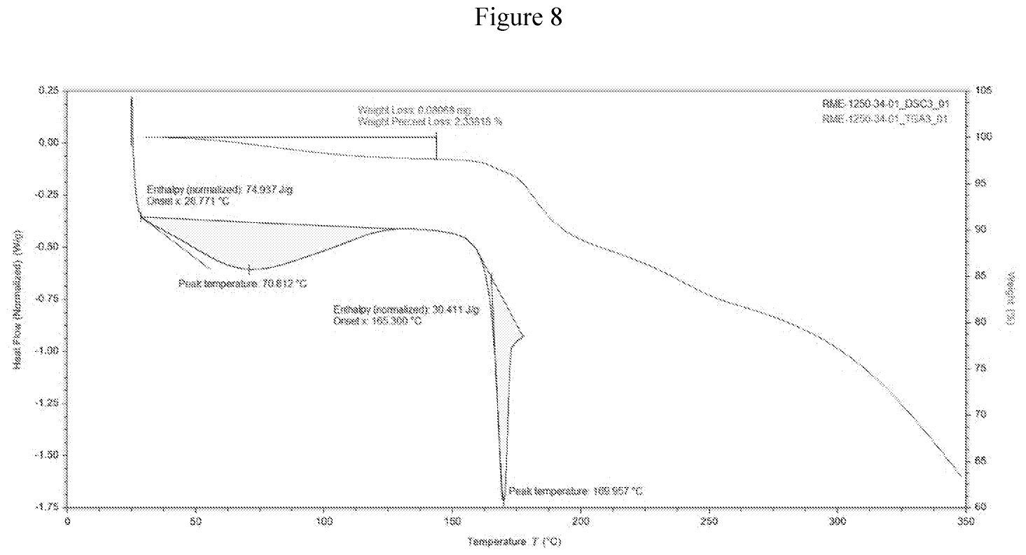

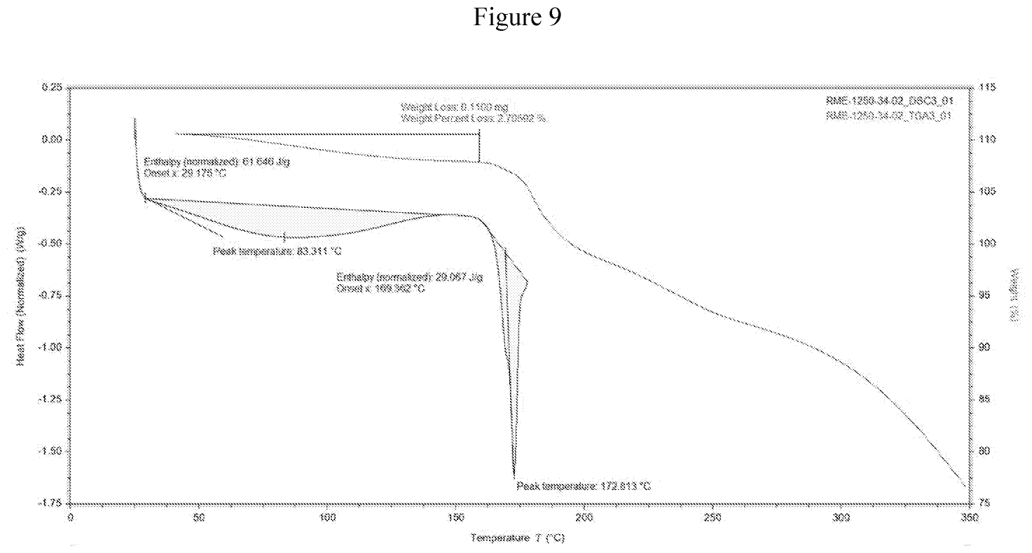

In one embodiment, the crystalline form of Compound 1-Na is Form A, wherein Form A is characterized by a DSC having a first onset temperature between about 27° C. and about 30° C., e.g., about 29° C., and a second onset temperature between about 159° C. and about 172° C., for example, 165 or 169° C. In one embodiment, the crystalline form of Compound 1-Na is Form A, wherein Form A is characterized by a DSC having a first onset temperature at approximately 29° C. and a second onset temperature at approximately 165° C. to 169° C. In one embodiment, the crystalline form of Compound 1-Na is Form A, wherein Form A is characterized by a DSC having a first onset temperature at approximately 29° C. and a second onset temperature at approximately 167° C. In one embodiment, the crystalline form of Compound 1-Na is Form A, wherein Form A is characterized by a DSC having a first onset temperature at approximately 29° C. and a second onset temperature at approximately 169° C. In one embodiment, the crystalline form of Compound 1-Na is Form A, wherein Form A is characterized by a DSC pattern substantially similar to that shown in FIG. 5, FIG. 8, or FIG. 9.

The low temperature endotherm onsets (e.g. 27-30° C.) correspond to the loss of non-crystal water from the solid (as evidenced by the corresponding weight loss shown via TGA, FIGS. 8 and 9). For example, a very broad endothermic signal that can be seen between about 29 and 140° C. (FIG. 9) correlates with the loss of weight in thermogravimetry (TR).

In one embodiment, the crystalline Form A is anhydrous.

In one embodiment, the crystalline Form A is thermally stable in the absence of solvents.

In one embodiment, the crystalline Form A is moderately hygroscopic below 80% relative humidity (RH) (e.g., about 25% RH, about 40% RH, about 50% RH, about 50% RH, about 60% RH, or about 70% RH). In one embodiment, the Form A polymorph is moderately hygroscopic between 40% RH and 80% RH.

“Moderately hygroscopic” indicates that the crystalline Form A absorbs less than 10% w/w water. In one embodiment, “moderately hygroscopic” indicates that the crystalline Form A absorbs less than about 9% w/w water. In one embodiment, “moderately hygroscopic” indicates that the crystalline Form A absorbs less than about 8% w/w water. In one embodiment, “moderately hygroscopic” indicates that the crystalline Form A absorbs less than about 7% w/w water. In one embodiment, “moderately hygroscopic” indicates that the crystalline Form A absorbs less than about 6% w/w water. In one embodiment, “moderately hygroscopic” indicates that the crystalline Form A absorbs less than about 5% w/w water. In one embodiment, “moderately hygroscopic” indicates that the crystalline Form A absorbs less than about 4% w/w water. In one embodiment, “moderately hygroscopic” indicates that the crystalline Form A absorbs between about 0% w/w and about 4% w/w water.

In one embodiment, the crystalline Form A is deliquescent at a humidity higher than about 80% RH.

In one embodiment, the crystalline Form A is anhydrous, thermally stable in the absence of solvents, and moderately hygroscopic below about 80% relative humidity (RH) (e.g., about 25% RH, about 40% RH, about 50% RH, about 50% RH, about 60% RH, or about 70% RH). In one embodiment, the crystalline Form A is anhydrous, thermally stable in the absence of solvents, and moderately hygroscopic below about 80% relative humidity (RH) (e.g., about 25% RH, about 40% RH, about 50% RH, about 50% RH, about 60% RH, or about 70% RH).

In one embodiment, the crystalline Form A is anhydrous, thermally stable in the absence of solvents, and moderately hygroscopic between about 40% RH and about 80% RH, but deliquescent at higher humidity (e.g., higher than about 80% RH).

In one embodiment, the crystalline Form A is stable for at least two weeks at about 25° C., about 60% RH. In one embodiment, the crystalline Form A is stable for at least 1 month at about 25° C., about 60% RH.

In one aspect, this application pertains to a method of preparing crystalline Form A of Compound 1-Na. In one embodiment, the method comprises:

(a) dissolving amorphous Compound 1-Na in a solvent to form a solution with or without heating;

(b) cooling the solution;

(c) repeating step (a) and step (b) for one or more times; and

(f) filtering the product from step (c) and drying the product under vacuum.

In one embodiment, amorphous Compound 1-Na is dissolved is one or more organic solvents or a mixture thereof. In one embodiment, amorphous Compound 1-Na is dissolved in acetonitrile.

In one embodiment, step (a) comprises heating Compound 1-Na in the solvent to facilitate the dissolution of Compound 1-Na. In one embodiment, step a comprises heating Compound 1-Na in the solvent to approximately 25° C., 30° C., 35° C., 40° C., 45° C., or 50° C. In one embodiment, step a comprises heating Compound 1-Na in the solvent to approximately 30° C.

In one embodiment, step (b) comprises cooling the solution comprising Compound 1-Na to approximately 18-25° C. In one embodiment, step (b) comprises cooling the solution comprising Compound 1-Na to about 20° C.

In one embodiment, step (c) is repeated once. In one embodiment, step (c) is repeated twice. In one embodiment, step (c) is repeated three times. In one embodiment, step (c) is repeated more than three times. In one embodiment, step (c) is repeated four times. In one embodiment, step (c) is repeated five times. In one embodiment, step (c) is repeated six times. In one embodiment, step (c) is repeated seven times. In one embodiment, step (c) is repeated eight times. In one embodiment, step (c) is repeated nine times. In one embodiment, step (c) is repeated ten times. In one embodiment, step (c) is repeated more than ten times. In one embodiment, step (c) is repeated more than twenty times. In one embodiment, step (c) is repeated thirteen times.

In one aspect, this application pertains to a method of preparing crystalline Form A of Compound 1-Na. In one embodiment, the method comprises:

(a) dissolving amorphous Compound 1-Na in a solvent to form a solution with or without heating;

(b) optionally cooling the solution comprising Compound 1-Na;

(c) adding a crystalline seed of the crystalline Form A of Compound 1-Na to the solution;

(d) adding acetonitrile to the solution;

(e) cooling the solution; and

(f) isolating the crystalline Form A of Compound 1-Na under vacuum filtration.

In one embodiment, amorphous Compound 1-Na is dissolved is one or more organic solvents or a mixture thereof. In one embodiment, amorphous Compound 1-Na is dissolved in acetonitrile. In one embodiment, amorphous Compound 1-Na is dissolved in a mixture of ethanol and acetonitrile.

In one embodiment, the ratio of the ethanol:acetonitrile is between about 80:20 and about 10:90. In one embodiment, the ratio of the ethanol:acetonitrile is about 80:20, about 70:30, about 60:40, about 50:50, about 40:60, about 30:70, about 20:80, or about 10:90. In one embodiment, the ratio of the ethanol:acetonitrile is about 60:40, about 50:50, about 40:60, about 30:70, or about 20:80. In one embodiment, the ratio of the ethanol:acetonitrile is about 40:60, about 30:70, or about 20:80. In one embodiment, the ratio of the ethanol:acetonitrile is about 30:70.

In one embodiment, the concentration of Compound 1-Na after dissolution in step (a) is about 0.01-0.5M. In one embodiment, the concentration of Compound 1-Na after dissolution in step (a) is about 0.01-0.1M. In one embodiment, the concentration of Compound 1-Na after dissolution in step (a) is about 0.1-0.2M. In one embodiment, the concentration of Compound 1-Na after dissolution in step (a) is about 0.2-0.3M. In one embodiment, the concentration of Compound 1-Na after dissolution in step (a) is about 0.3-0.4M. In one embodiment, the concentration of Compound 1-Na after dissolution in step (a) is about 0.4-0.5M. In one embodiment, the concentration of Compound 1-Na after dissolution in step (a) is about 0.10, about 0.11, about 0.12, about 0.13, about 0.14, about 0.15, about 0.16, about 0.17, about 0.18, about 0.19, or about 0.20M. In one embodiment, the concentration of Compound 1-Na after dissolution in step (a) is about 0.15, about 0.16, about 0.17, about 0.18, about 0.19, or about 0.20M.

In one embodiment, the dissolution of amorphous Compound 1-Na in step (a) is conducted at about 10-about 40° C. In one embodiment, the dissolution of amorphous Compound 1-Na in step (a) is conducted at about 15-about 35° C. In one embodiment, dissolution of amorphous Compound 1-Na in step (a) is conducted at about 20-about 30° C. In one embodiment, dissolution of amorphous Compound 1-Na in step (a) is conducted at approximately 20° C. In one embodiment, dissolution of amorphous Compound 1-Na in step (a) is conducted at approximately 25° C. In one embodiment, dissolution of amorphous Compound 1-Na in step (a) is conducted at approximately 30° C.

In one embodiment, step a comprises heating Compound 1-Na in the solvent to facilitate the dissolution of Compound 1-Na. In one embodiment, step (a) comprises heating Compound 1-Na in the solvent to approximately 25° C., 30° C., 35° C., 40° C., 45° C., or 50° C. In one embodiment, step (a) comprises heating Compound 1-Na in the solvent to approximately 30° C.

In one embodiment, this application pertains to a method of preparing crystalline Form A of Compound 1-Na optionally comprising step (b), wherein the solution comprising Compound 1-Na is cooled to approximately 20° C. In one embodiment, this application pertains to a method of preparing crystalline Form A of Compound 1-Na comprising step (b), wherein the solution comprising Compound 1-Na is cooled to approximately 20° C.

In one embodiment, the application pertains to a method of preparing crystalline Form A of Compound 1-Na comprising step (c), wherein a crystalline seed of the crystalline Form A of Compound 1-Na is added to the solution comprising Compound 1-Na. In one embodiment, the amount of the seed added to the solution is about 0.1, about 0.2, about 0.3, about 0.4, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, or about 3.0% by mass of the amount of amorphous Compound 1-Na dissolved in step (a). In one embodiment, the amount of the seed added to the solution is about 0.1, about 0.2, about 0.3, about 0.4, about 0.5, about 0.6, about 0.7, about 0.8, about 0.9, about 1.0, about 1.1, about 1.2, about 1.3, about 1.4, or about 1.5% by mass of the amount of amorphous Compound 1-Na dissolved in step (a). In one embodiment, the amount of the seed added to the solution is about 0.1, about 0.2, about 0.3, about 0.4, about 0.5, about 0.6, about 0.7, about 0.8, about 0.9, or about 1.0% by mass of the amount of amorphous Compound 1-Na dissolved in step (a). In one embodiment, the amount of the seed added to the solution is about 0.3, about 0.4, about 0.5, about 0.6, or about 0.7% by mass of the amount of amorphous Compound 1-Na dissolved in step (a). In one embodiment, the amount of the seed added to the solution is about 0.4, about 0.5, or about 0.6% by mass of the amount of amorphous Compound 1-Na dissolved in step (a). In one embodiment, the amount of the seed added to the solution is about 0.5% by mass of the amount of amorphous Compound 1-Na dissolved in step (a).

In one embodiment, this application pertains to a method of preparing the crystalline Form A of Compound 1-Na comprising step (d), wherein acetonitrile is added to the solution. In one embodiment, the ratio, by volume, of the amount of acetonitrile added to the solution to the amount of solvent used in step (a) is about 0.3, about 0.4, about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, or about 3.0. In one embodiment, the ratio, by volume, of the amount of acetonitrile added to the solution in step (d) to the amount of solvent used in step (a) is about 0.7, about 0.8, about 0.9, about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, or about 2.6. In one embodiment, the ratio, by volume, of the amount of acetonitrile added to the solution to the amount of solvent used in step (a) is about 0.9, about 1.0, about 1.1, about 1.2, about 1.3, about 1.4, or about 1.5. In one embodiment, the ratio, by volume, of the amount of acetonitrile added to the solution to the amount of solvent used in step (a) is about 1.0, about 1.1, about 1.2, about 1.3, or about 1.4. In one embodiment, the ratio, by volume, of the amount of acetonitrile added to the solution to the amount of solvent used in step (a) is about 1.1, about 1.2, or about 1.3. In one embodiment, the ratio, by volume, of the amount of acetonitrile added to the solution to the amount of solvent used in step (a) is about 1.2.

In one embodiment, this application pertains to a method of preparing the crystalline Form A of Compound 1-Na comprising step (e), wherein the solution is cooled. In one embodiment, the solution is cooled to about −15° C.-15° C. at about 0.1-0.5° C./min and stirred at this temperature for an additional 4-24 hours. In one embodiment, the solution is cooled to about −10° C.-10° C. at about 0.1-0.5° C./min and stirred at this temperature for an additional about 4-24 hours. In one embodiment, the solution is cooled to about 0° C.-10° C. at about 0.1-0.5° C./min and stirred at this temperature for an additional about 4-24 hours. In one embodiment, the solution is cooled to about 5° C. at about 0.1-0.5° C./min and stirred at this temperature for an additional about 4-24 hours. In one embodiment, the solution is cooled to about 5° C. at about 0.1° C./min and stirred at this temperature for an additional about 8, about 9, about 10, about 11, about 12, about 13, about 14, about 15, or about 16 hours. In one embodiment, the solution is cooled to about 5° C. at about 0.1° C./min and stirred at this temperature for an additional about 10, about 11, about 12, about 13, or about 14 hours. In one embodiment, the solution is cooled to about 5° C. at about 0.1° C./min and stirred at this temperature for an additional about 11, about 12, or about 13 hours. In one embodiment, the solution is cooled to about 5° C. at about 0.1° C./min and stirred at this temperature for an additional about 12 hours.

In one embodiment, this application pertains to a method of preparing the crystalline Form A of Compound 1-Na comprising step f, wherein the crystalline Form A of Compound 1-Na is isolated under vacuum filtration.

In one embodiment, this application pertains to a method of preparing the crystalline Form A of Compound 1-Na comprising step f, wherein the crystalline Form A of Compound 1-Na is isolated under vacuum filtration, and then optionally air-dried.

In one embodiment, this application pertains to a method of preparing the crystalline Form A of Compound 1-Na comprising step f, wherein the crystalline Form A of Compound 1-Na is isolated under vacuum filtration, and then air-dried. In one embodiment, the crystalline Form A of Compound 1-Na is isolated under vacuum filtration, and then air-dried for 1-100 minutes. In one embodiment, the crystalline Form A of Compound 1-Na is air-dried for about 1-90 minutes, about 5-75 minutes, about 10-60 minutes, about 15-45 minutes, or about 20-30 minutes. In one embodiment, the crystalline Form A of Compound 1-Na is air-dried for about 10-60 minutes.

In one embodiment, this application pertains to a pharmaceutical composition comprising a crystalline form of Compound 1-Na (i.e., Form A), and a pharmaceutically acceptable diluent, excipient, or carrier.

As used herein, the term “Compound 1” refers to

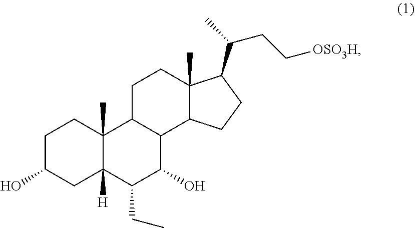

(6α-ethyl-3α,7α,23-trihydroxy-24-nor-5β-cholan-23-hydrogen sulphate).

As used herein, the term “Compound 1-Na” or “1-Na” refers to

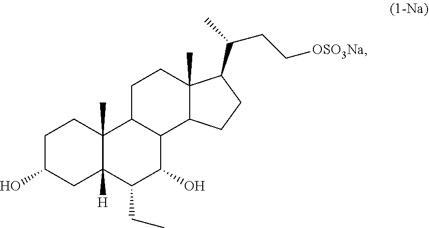

(6α-ethyl-3α,7α,23-trihydroxy-24-nor-5β-cholan-23-sulphate sodium),
   or the sodium salt of Compound 1.

In one of the embodiments Compound 1-OH is an intermediate compound in the synthesis of Compound 1 and Compound 1-Na.

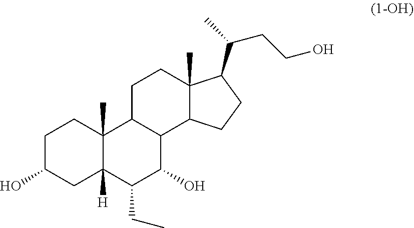 [SMILES: CC[C@H]1[C@@H](O)C2C3CC[C@H]([C@H](C)CCO)[C@@]3(C)CCC2[C@@]2(C)CC[C@@H](O)C[C@@H]12]

Various data, e.g., XRPD, obtained for crystalline form of C23 alcohol analog (triol) of Compound 1 or Compound 1-Na, i.e., Compound 1-OH, can be used for validation of data for Compound 1-Na and its crystalline Form A.

Synthesis of Compound 1 or Compound 1-Na

Compound 1 or Compound 1-Na can be prepared by methods known in the art, e.g., those described in U.S. Pat. No. 7,932,244 and US 2015-0291653, the entire contents of each of which are incorporated herein by reference.

Standard synthetic methods and procedures for the preparation of organic molecules and functional group transformations and manipulations, including the use of protective groups, can be obtained from the relevant scientific literature or from standard reference textbooks in the field. Although not limited to any one or several sources, recognized reference textbooks of organic synthesis include: Smith, M. B.; March, J. March's Advanced Organic Chemistry: Reactions, Mechanisms, and Structure, 5^{th} ed.; John Wiley & Sons: New York, 2001; and Greene, T. W.; Nuts, P. G. M. Protective Groups in Organic Synthesis, 3^{td}; John Wiley & Sons: New York, 1999.

The terms “crystalline polymorph”, “crystal polymorph”, “crystal form”, “polymorph”, or “polymorphic form” or “crystalline form” means crystal structures in which a compound (e.g., Compound 1-Na) can crystallize in different crystal packing arrangements, all of which have the same elemental composition. Different crystalline forms usually have different X-ray diffraction patterns, infrared spectra, melting points, density, crystal shape, optical and electrical properties, stability and solubility. Crystallization solvent, rate of crystallization, storage temperature, and other factors may cause one crystal form to dominate. Different crystalline forms or polymorphs may have different physical properties such as, for example, melting temperatures, heats of fusion, solubilities, dissolution rates and/or vibrational spectra as a result of the arrangement or conformation of the molecules in the crystal lattice.

The differences in physical properties exhibited by crystalline forms or polymorphs affect pharmaceutical parameters such as storage stability, compressibility and density (important in formulation and product manufacturing), and dissolution rates (an important factor in bioavailability). Differences in stability can also result from changes in chemical reactivity (e.g., differential oxidation, such that a dosage form discolors more rapidly when comprised of one polymorph or crystalline form than when comprised of another polymorph or crystalline form) or mechanical property (e.g., tablets crumble on storage as a kinetically favored crystalline from or polymorph converts to thermodynamically more stable crystalline form or polymorph) or both (e.g., tablets of one polymorph are more susceptible to breakdown at high humidity). As a result of solubility/dissolution differences, in the extreme case, some crystalline or polymorphic transitions may result in lack of potency or, at the other extreme, toxicity. In addition, the physical properties of the crystal may be important in processing, for example, one crystalline form or polymorph might be more likely to form solvates or might be difficult to filter and wash free of impurities (e.g., particle shape and size distribution might be different between crystalline forms or polymorphs).

Techniques for characterizing crystalline forms or polymorphs include, but are not limited to, differential scanning calorimetry (DSC), X-ray powder diffractometry (XRPD), single crystal X-ray diffractometry, vibrational spectroscopy (e.g., IR and Raman spectroscopy), TGA (Thermogravimetric analysis), DTA (Differential thermal analysis), DVS (Dynamic vapour sorption), solid state NMR, hot stage optical microscopy, scanning electron microscopy (SEM), electron crystallography and quantitative analysis, particle size analysis (PSA), surface area analysis, solubility studies, and dissolution studies.

As used herein, the term “amorphous form” refers to a noncrystalline solid state form of a substance.

The term “treating” as used herein refers to any indicia of success in the treatment or amelioration of a disease or disorder. Treating can include, for example, reducing or alleviating the severity of one or more symptoms of a disease or disorder, or it can include reducing the frequency with which symptoms of a disease or disorder are experienced by a patient “Treating” can also refer to reducing or eliminating a condition of a part of the body, such as a cell, tissue or bodily fluid (e.g., blood).

As used herein, the term “preventing” refers to the partial or complete prevention of a, disease or disorder in an individual or in a population, or in a part of the body, such as a cell, tissue or bodily fluid (e.g., blood), The term “prevention” does not establish a requirement for complete prevention of a disease or disorder in the entirety of the treated population of individuals or cells, tissues or fluids of individuals.

The term “treat or prevent” is used herein to refer to a method that results in some level of treatment or amelioration of a disease or disorder, and contemplates a range of results directed to that end, including; but not restricted to prevention of a disease or disorder entirely.

The term “therapeutically effective amount” or “effective amount”, as used herein, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician. In a preferred aspect, the disease or disorder to be treated or prevented is a FXR-mediated disease or disorder.

For any compound, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary depending upon various factors, including but not limited to the dosage form employed, sensitivity of the patient, and the route of administration.

As used herein, “pharmaceutically acceptable” refers to a material that is not biologically or otherwise undesirable, e.g., the material may be incorporated into a pharmaceutical composition administered to a patient without causing any significant undesirable biological effects or interacting in a deleterious manner with any of the other components of the composition in which it is contained.

“Pharmaceutically acceptable diluent/excipient/carrier” means a diluent/excipient/carrier that is useful in preparing a pharmaceutical composition that is generally safe, non-toxic, and neither biologically nor otherwise undesirable, and is acceptable for veterinary use as well as human pharmaceutical use. A “pharmaceutically acceptable diluent/excipient/carrier” as used in the specification and claims includes both one and more than one such diluent/excipient/carrier.

Pharmaceutically acceptable carriers, for example, or excipients have met the required standards of toxicological and manufacturing testing and/or are included on the Inactive Ingredient Guide prepared by the U.S. Food and Drug administration. As used herein, the term “solvate” means solvent addition form or forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate, when the solvent is alcohol, the solvate formed is an alcoholate. Hydrates are formed by the combination of one or more molecules of water with one of the substances in which the water retains its molecular state as H₂O, such combination being able to form one or more hydrates. Compound 1-Na of the present application may exist in either hydrated or unhydrated (the anhydrous) form or as solvate with other solvent molecule(s) or in an unsolvated form. Nonlimiting examples of hydrates include monohydrates, dihydrates, etc. Nonlimiting examples of solvates include DCM (dichloromethane) solvates, MEK (methylethyl ketone) solvates, THF (tetrahydrofuran) solvates, etc.

As used herein, the terms “unsolvated” or “desolvated” refer to a solid state form (e.g., crystalline forms, amorphous forms, and mesomorphs) of a substance which does not contain solvent.

As used herein, a compound is “stable” where significant amounts of degradation products are not observed under constant conditions of humidity (e.g., about 10%, about 20%, about 30%, about 40%, about 50%, about 60%, about 70%, about 75%, about 80%, about 85%, about 90%, and about 95% RH), light exposure, and/or temperatures (e.g., higher than about 0° C., e.g., about 20° C., about 25° C., about 30° C., about 35° C., about 40° C., about 45° C., about 50° C., about 55° C., about 60° C., about 65° C., and about 70° C.) over a certain period (e.g., one week, two weeks, three weeks, and four weeks). A compound is not considered to be stable at a certain condition when degradation impurities appear or an area percentage (e.g., AUC as characterized by HPLC) of existing impurities begins to grow. The amount of degradation growth as a function of time is important in determining compound stability.

As used herein, the term “mixing” means combining, blending, stirring, shaking, swirling, or agitating. The term “stirring” as used herein can mean mixing, shaking, agitating, or swirling. The term “agitating” as used herein can mean mixing, shaking, stirring, or swirling.

Unless explicitly indicated otherwise, the terms “approximately” and “about” are synonymous. In one embodiment, “approximately” and “about” refer to recited amount, value, or duration, e.g, ±20%, ±15%, ±10%, ±8%, ±6%, ±5%, ±4%, ±2%, ±1%, or ±0.5% of that value. In another embodiment, “approximately” and “about” refer to listed amount, value, or duration ±10%, ±8%, ±6%, ±5%, ±4%, or ±2%. In yet another embodiment, “approximately” and “about” refer to listed amount, value, or duration ±5%. In yet another embodiment, “approximately” and “about” refer to listed amount, value, or duration ±2%.

When the terms “approximately” and “about” are used when reciting XRPD peaks, these terms refer to the recited X-ray powder diffraction peak ±0.3° 2θ (theta), ±0.2° 2θ (theta), or ±0.1° 2θ (theta). In another embodiment, the terms “approximately” and “about” refer to the listed X-ray powder diffraction peak ±0.2° 2θ (theta). In another embodiment, the terms “approximately” and “about” refer to the listed X-ray powder diffraction peak ±0.1° 2θ (theta).

When the terms “approximately” and “about” are used when reciting temperature or temperature range, these terms refer to the recited temperature or temperature range ±5° C., ±2° C., or ±1° C. In another embodiment, the terms “approximately” and “about” refer to the recited temperature or temperature range ±2° C. In another embodiment, the terms “approximately” and “about” refer to the recited temperature or temperature range ±1° C.

A “disease or disorder in which FXR plays a role” or “FXR-mediated disease or disorder” refers to a disease or disorder in which modulation of FXR (e.g., activation of FXR) is involved in the initiation and/or development of the disease or disorder, and/or can be used in the treatment and/or prevention of the disease or disorder. In one embodiment, “a disease or disorder in which FXR plays a role” or “FXR-mediated disease or disorder” is cardiovascular disease, e.g., atherosclerosis, arteriosclerosis, hypercholesteremia, or hyperlipidemia, chronic liver disease, gastrointestinal disease, renal disease, metabolic disease, cancer (e.g., colorectal cancer, hepatocellular carcinoma), or neurological indications or disorders such as stroke.

In one embodiment, the chronic liver disease is primary biliary cirrhosis (PBC), cerebrotendinous xanthomatosis (CTX), primary sclerosing cholangitis (PSC), drug induced cholestasis, intrahepatic cholestasis of pregnancy, parenteral nutrition associated cholestasis (PNAC), bacterial overgrowth or sepsis associated cholestasis, autoimmune hepatitis, chronic viral hepatitis, alcoholic liver disease, nonalcoholic fatty liver disease (NAFLD), nonalcoholic steatohepatitis (NASH), liver transplant associated graft versus host disease, living donor transplant liver regeneration, congenital hepatic fibrosis, choledocholithiasis, granulomatous liver disease, intra- or extrahepatic malignancy, Sjogren's syndrome, Sarcoidosis, Wilson's disease, Gaucher's disease, hemochromatosis, or alpha 1-antitrypsin deficiency.

In one embodiment, the gastrointestinal disease is inflammatory bowel disease (IBD) (including Crohn's disease and ulcerative colitis), irritable bowel syndrome (IBS), bacterial overgrowth, malabsorption, post-radiation colitis, or microscopic colitis.

In one embodiment, the renal disease is diabetic nephropathy, focal segmental glomerulosclerosis (FSGS), hypertensive nephrosclerosis, chronic glomerulonephritis, chronic transplant glomerulopathy, chronic interstitial nephritis, or polycystic kidney disease.

In one embodiment, the cardiovascular disease is atherosclerosis, arteriosclerosis, dyslipidemia, hypercholesterolemia, or hypertriglyceridemia.

In one embodiment, the metabolic disease is insulin resistance, Type I and Type II diabetes, or obesity.

General Types of Recrystallization Procedures

In one aspect, this application pertains to a method of preparing the crystalline Form A of Compound 1-Na from an amorphous form of Compound 1-Na.

In one embodiment, this application pertains to a method of preparing the crystalline Form A of Compound 1-Na by crystallization.

In one embodiment, the crystallization of the crystalline Form A of Compound 1-Na can be performed under slow evaporation conditions, e.g., the amorphous form of Compound 1-Na is dissolved in relevant solvents at about 18-27° C., e.g., 25° C., followed by cooling at about 0 to 10° C., e.g., 5° C., and removing the lids to allow evaporation under a N₂ flow at about 0 to 10° C., e.g., 5° C., before analyzing by XRPD.

In one embodiment, the crystallization of the crystalline Form A of Compound 1-Na can be performed under slow cooling conditions, e.g., the amorphous form of Compound 1-Na is dissolved in relevant solvents at about 20-35° C., e.g., 30° C., followed by cooling to about 0 to 10° C., e.g., 5° C. at about 0.05 to 0.30° C./min, e.g. 0.1° C./min, and stirring at this temperature for about 10-30 hours, e.g., 16 hours. Solids are then filtered, air-dried, and analyzed by XRPD.

In one embodiment, the crystallization of the crystalline Form A of Compound 1-Na can be performed under antisolvent addition conditions, e.g., the amorphous form of Compound 1-Na is dissolved in a solvent system at about 25° C., and the resulted solution is then treated with antisolvent (e.g., acetonitrile or n-heptane) added dropwise until the solution becomes cloudy. The turbid solutions were cooled to about 5° C. for about 16 hours. The solids are filtered and dried under by vacuum filtration for about 20 min, and the residues are initially analyzed by XRPD.

In one embodiment, the crystallization of the crystalline Form A of Compound 1-Na can be performed under maturation in neat solvents, e.g., the amorphous form of Compound 1-Na is suspended in the relevant solvent at two different concentrations, about 50 vol (20 mg/mL) and about 200 vol (5 mg/mL) at about 50° C. The suspensions are shaken in the maturation chamber at between about 25-50° C. for about 3 days (5-15, e.g., 8 h cycles), then allowed to stand at room temperature for about 10 min. After the maturation treatment, the mother liquors of all the samples are taken and placed at about 5° C. Any residual solids in sufficient amount are filtered and analyzed by XRPD.

In one embodiment, the crystallization of the crystalline Form A of Compound 1-Na can be performed under maturation in solvent mixtures, e.g., the amorphous form of Compound 1-Na is suspended in the relevant solvent system at two different concentrations, about 50 vol (20 mg/mL) and about 200 vol (5 mg/mL) at about 50° C. The suspensions were shaken in the maturation chamber between about 25-50° C. for about 3 days (8 h cycles), then allowed to stand at room temperature for about 10 min. The residual solids were filtered, air-dried and analyzed by XRPD.

In one embodiment, the crystallization of the crystalline Form A of Compound 1-Na can be performed under maturation, e.g., the amorphous form of Compound 1-Na is suspended in the relevant solvents (50 vol). The suspensions are heated to about 30° C. at about 0.5° C./min and stirred at this temperature for about 1 hour. The suspensions were then cooled to about 0° C. at about 0.2° C./min and again stirred at this temperature for about 1 hour. This process was repeated until about 8 heating/cooling cycles are completed. Then, the samples are allowed to stand at room temperature for about 10 min. The residual solids are filtered, air-dried and analyzed by XRPD.

These example procedures and conditions are not intended to be limiting.

### Methods of the Application

This application pertains to a method of treating or preventing an FXR-mediated disease or disorder in a subject in need thereof, comprising administering a therapeutically effective amount of a crystalline form of Compound 1-Na (i.e., Form A) or a pharmaceutical composition comprising a crystalline form of Compound 1-Na (i.e., Form A).

In one aspect, this application pertains to a crystalline form of Compound 1-Na (i.e., Form A) or a pharmaceutical composition comprising a crystalline form of Compound 1-Na (i.e., Form A) for treating or preventing an FXR-mediated disease or disorder.

In one aspect, this application pertains to the use of a crystalline form of Compound 1-Na (i.e., Form A) or a pharmaceutical composition comprising a crystalline form of Compound 1-Na (i.e., Form A) in the manufacture of a medicament for treating or preventing an FXR-mediated disease or disorder.

In one embodiment, the present disclosure relates to a method of treating or preventing an FXR-medated disease or disorder in a subject in need thereof, wherein an FXR-mediated disease or disorder is cardiovascular disease or disorder, e.g., atherosclerosis, arteriosclerosis, hypercholesteremia, or hyperlipidemia, chronic liver disease or disorder, gastrointestinal disease or disorder, renal disease or disorder, metabolic disease or disorder, cancer (e.g., colorectal cancer), or neurological disease or disorder, e.g., stroke.

In one aspect, this application pertains to a crystalline form of Compound 1-Na (i.e., Form A) or a pharmaceutical composition comprising a crystalline form of Compound 1-Na (i.e., Form A) for treating or preventing an FXR-mediated disease or disorder, wherein the FXR-mediated disease or disorder is cardiovascular disease or disorder, for example, atherosclerosis, arteriosclerosis, hypercholesteremia, or hyperlipidemia, chronic liver disease or disorder, gastrointestinal disease or disorder, renal disease or disorder, metabolic disease or disorder, cancer (e.g., colorectal cancer), or neurological disease or disorder, e.g. stroke.

In one embodiment, the present disclosure relates to a method of treating or preventing an FXR-medated disease or disorder in a subject in need thereof, wherein an FXR-mediated disease or disorder is the chronic liver disease is primary biliary cirrhosis (PBC), cerebrotendinous xanthomatosis (CTX), primary sclerosing cholangitis (PSC), drug induced cholestasis, intrahepatic cholestasis of pregnancy, parenteral nutrition associated cholestasis (PNAC), bacterial overgrowth or sepsis associated cholestasis, autoimmune hepatitis, chronic viral hepatitis, alcoholic liver disease, nonalcoholic fatty liver disease (NAFLD), nonalcoholic steatohepatitis (NASH), liver transplant associated graft versus host disease, living donor transplant liver regeneration, congenital hepatic fibrosis, choledocholithiasis, granulomatous liver disease, intra- or extrahepatic malignancy, Sjogren's syndrome, Sarcoidosis, Wilson's disease, Gaucher's disease, hemochromatosis, or alpha 1-antitrypsin deficiency.

In one embodiment, the present disclosure relates to a method of treating or preventing an FXR-medated disease or disorder in a subject in need thereof, wherein an FXR-mediated disease or disorder is a gastrointestinal disease, wherein the gastrointestinal disease or disorder is inflammatory bowel disease (IBD) (including Crohn's disease and ulcerative colitis), irritable bowel syndrome (IBS), bacterial overgrowth, malabsorption, post-radiation colitis, or microscopic colitis.

In one embodiment, the present disclosure relates to a method of treating or preventing an FXR-medated disease or disorder in a subject in need thereof, wherein an FXR-mediated disease or disorder is a renal disease or disorder, wherein the renal disease or disorder is diabetic nephropathy, focal segmental glomerulosclerosis (FSGS), hypertensive nephrosclerosis, chronic glomerulonephritis, chronic transplant glomerulopathy, chronic interstitial nephritis, or polycystic kidney disease.

In one embodiment, the present disclosure relates to a method of treating or preventing an FXR-medated disease or disorder in a subject in need thereof, wherein an FXR-mediated disease or disorder is a cardiovascular disease or disorder, wherein the cardiovascular disease or disorder, is atherosclerosis, arteriosclerosis, dyslipidemia, hypercholesterolemia, or hypertriglyceridemia.

In one embodiment, the present disclosure relates to a method of treating or preventing an FXR-medated disease or disorder in a subject in need thereof, wherein an FXR-mediated disease or disorder is a metabolic disease or discorder, wherein the metabolic disease or disorder is insulin resistance, Type I and Type II diabetes, or obesity.

In one aspect, this application pertains to a method of modulating FXR (e.g., activating FXR) in a subject in need thereof, comprising administering a therapeutically effective amount of a crystalline form of Compound 1-Na (i.e., Form A) or a pharmaceutical composition comprising a crystalline form of Compound 1-Na (i.e., Form A).

In one aspect, this application pertains to a crystalline form of Compound 1-Na (i.e., Form A) or a pharmaceutical composition comprising a crystalline form of Compound 1-Na (i.e., Form A) for modulating FXR (e.g., activating FXR).

In one aspect, this application pertains to use of a crystalline form of Compound 1-Na (i.e., Form A) or a pharmaceutical composition comprising a crystalline form of Compound 1-Na (i.e., Form A) in the manufacture of a medicament for modulating FXR (e.g., activating FXR).

### Pharmaceutical Compositions

A “pharmaceutical composition” is a formulation containing an active agent (e.g., a crystalline form of Compound 1-Na (i.e., Form A)) in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler, or a vial. The quantity of active ingredient (e.g., a crystalline form of Compound 1-Na (i.e., Form A)) in a unit dose of composition is an effective amount and is varied according to the particular treatment involved.

The present application provides pharmaceutical compositions comprising a crystalline form of Compound 1-Na (i.e., Form A), and a pharmaceutically acceptable diluent, excipient, or carrier. The pharmaceutical composition of the present disclosure can be administered enternally, orally, transdermally, pulmonarily, inhalationally, buccally, sublingually, intraperintoneally, subcutaneously, intramuscularly, intravenously, rectally, intrapleurally, intrathecally, intranasally, parenterally, or topically.

In particular, tablets, coated tablets, capsules, syrups, suspensions, drops or suppositories are used for enteral administration, solutions, preferably oily or aqueous solutions, furthermore suspensions, emulsions or implants, are used for parenteral administration, and ointments, creams or powders are used for topical application. Suitable dosage forms include, but are not limited to capsules, tablets, pellets, dragees, semi-solids, powders, granules, suppositories, ointments, creams, lotions, inhalants, injections, cataplasms, gels, tapes, eye drops, solution, syrups, aerosols, suspension, emulsion, which can be produced according to methods known in the art, for example as described below:

tablets: mixing of active ingredient/sand auxiliaries, compression of said mixture into tablets (direct compression), optionally granulation of part of mixture before compression.

capsules: mixing of active ingredient/s and auxiliaries to obtain a flowable powder, optionally granulating powder, filling powders/granulate into opened capsules, capping of capsules.

semi-solids (ointments, gels, creams): dissolving/dispersing active ingredient/s in an aqueous or fatty carrier; subsequent mixing of aqueous/fatty phase with complementary fatty/aqueous phase, homogenization (creams only).

suppositories (rectal and vaginal): dissolving/dispersing active ingredient/sin carrier material liquified by heat (rectal: carrier material normally a wax; vaginal: carrier normally a heated solution of a gelling agent), casting said mixture into suppository forms, annealing and withdrawal suppositories from the forms.

aerosols: dispersing/dissolving active agent/sin a propellant, bottling said mixture into an atomizer.

Suitable formulations for parenteral administration include aqueous solutions of the active compounds in watersoluble form, for example, water-soluble salts and alkaline solutions. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides or polyethylene glycol-400 (the compounds are soluble in PEG-400). Aqueous injection suspensions may contain substances, which increase the viscosity of the suspension, including, for example, sodium carboxymethyl cellulose, sorbitol, and/or dextran, optionally, the suspension may also contain stabilizers. For administration as an inhalation spray, it is possible to use sprays in which the active ingredient is either dissolved or suspended in a propellant gas or propellant gas mixture (for example CO₂ or chlorofluorocarbons). The active ingredient is advantageously used here in micronized form, in which case one or more additional physiologically acceptable solvents may be present, for example ethanol. Inhalation solutions can be administered with the aid of conventional inhalers. In addition, stabilizers may be added.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Dosage forms for the topical or transdermal administration include but are not limited to powders, sprays, ointments, pastes, creams, lotions, gels, solutions, patches and inhalants. In one embodiment, the active ingredient is mixed under sterile conditions with a pharmaceutically acceptable carrier, and with any preservatives, buffers or propellants that are required.

Suitable excipients are organic or inorganic substances, which are suitable for enteral (for example oral), parenteral or topical administration and do not react with the products of the disclosure, for example water, vegetable oils, benzyl alcohols, alkylene glycols, polyethylene glycols, glycerol triacetate, gelatine, carbohydrates, such as lactose, sucrose, mannitol, sorbitol or starch (maize starch, wheat starch, rice starch, potato starch), cellulose preparations and/or calcium phosphates, for example tricalcium phosphate or calcium hydrogen phosphate, magnesium stearate, talc, gelatine, tragacanth, methyl cellulose, hydroxypropylmethylcellulose, sodium carboxymethylcellulose, polyvinyl pyrrolidone and/or vaseline. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries include, without limitation, flow-regulating agents and lubricants, for example, silica, talc, stearic acid or salts thereof, such as magnesium stearate or calcium stearate, and/or polyethylene glycol.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, or sodium chloride in the composition. Prolonged absorption of the injectable compositions can be brought about by including in the composition an agent which delays absorption, for example, aluminum monostearate and gelatin. Sterile injectable solutions can be prepared by incorporating the active ingredient in the required amount in an appropriate solvent with one or a combination of ingredients enumerated above, as required, followed by filtered sterilization. Generally, dispersions are prepared by incorporating the active ingredient into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof. The compounds of the disclosure can be used, for example, for the production of injection preparations. The preparations indicated can be sterilized and/or can contain excipients such as lubricants, preservatives, stabilizers and/or wetting agents, emulsifiers, salts for affecting the osmotic pressure, buffer substances, colorants, flavourings and/or aromatizers. They can, if desired, also contain one or more further active compounds, e.g. one or more vitamins.

For administration by inhalation, the active ingredient is delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active ingredient is formulated into ointments, salves, gels, or creams as generally known in the art.

One skilled in the art will appreciate that it is sometimes necessary to make routine variations to the dosage depending on, for example, the age and condition of the patient. The dosage will also depend on the route of administration.

One skilled in the art will recognize the advantages of certain routes of administration. The dosage administered will be dependent upon the age, health, and weight of recipient, kind of concurrent treatment, if any, frequency of treatment, and the nature of the effect desired.

In one embodiment, the pharmaceutical composition of the present application is administered orally.

Oral compositions generally include an inert diluent or an edible pharmaceutically acceptable carrier. They can be enclosed in gelatin capsules or compressed into tablets. For the purpose of oral therapeutic administration, the active ingredient can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring. For example, oral compositions can be tablets or gelatin capsules comprising the active ingredient together with a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets also c) binders, e.g., magnesium aluminum silicate, starch paste, gelatin, tragacanth, methylcellulose, sodium carboxymethylcellulose and or polyvinylpyrrolidone; if desired d) disintegrants, e.g., starches, agar, alginic acid or its sodium salt, or effervescent mixtures; and/or e) absorbents, colorants, flavors and sweeteners.

Dragee cores are provided with suitable coatings, which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures.

In order to produce dosage form coatings resistant to gastric juices or to provide a dosage form affording the advantage of prolonged action (modified release dosage form), the tablet, dragee or pill can comprise an inner dosage and an outer dosage component me latter being in the form of an envelope over the former. The two components can be separated by an enteric layer, which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, acetyl alcohol, solutions of suitable cellulose preparations such as acetyl-cellulose phthalate, cellulose acetate or hydroxypropylmethyl-cellulose phthalate, are used. Dye stuffs or pigments may be added to the tablets or dragee coatings, for example, for identification or in order to characterize combinations of active compound doses. Suitable carrier substances are organic or inorganic substances which are suitable for enteral (e.g. oral) or parenteral administration or topical application and do not react with the compounds of disclosure, for example water, vegetable oils, benzyl alcohols, polyethylene glycols, gelatin, carbohydrates such as lactose or starch, magnesium stearate, talc and petroleum jelly.

Other pharmaceutical preparations, which can be used orally include push-fit capsules made of gelatine, as well as soft, sealed capsules made of gelatine and a plasticizer such as glycerol or sorbitol. The push-fit capsules can contain the active compounds in the form of granules, which may be mixed with fillers such as lactose, binders such as starches, and/or lubricants such as talc or magnesium stearate and, optionally, stabilizers. In soft capsules, the active compounds are preferably dissolved or suspended in suitable liquids, such as fatty oils, or liquid paraffin.

The liquid forms in which the compositions of the present disclosure may be incorporated for administration orally include aqueous solutions, suitably flavoured syrups, aqueous or oil suspensions, and flavoured emulsions with edible oils such as cottonseed oil, sesame oil, coconut oil or peanut oil, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, polyvinyl-pyrrolidone or gelatine.

Dosage forms for oral administration comprise modified release formulations. The term “immediate release” is defined as a release of the crystalline form of Compound 1-Na (i.e., Form A) from a dosage form in a relatively brief period of time, generally up to about 60 minutes. The term “modified release” is defined to include delayed release, extended release, and pulsed release. The term “pulsed release” is defined as a series of releases of drug from a dosage form. The term “sustained release” or “extended release” is defined as continuous release of the crystalline form of Compound 1-Na (i.e., Form A) from a dosage form over a prolonged period of time.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active ingredient calculated to produce the desired therapeutic effect in association with the required pharmaceutical carrier. The specification for the dosage unit forms of the application are dictated by and directly dependent on the unique characteristics of the active ingredient and the particular therapeutic effect to be achieved. In therapeutic applications, the dosages of the pharmaceutical compositions used in accordance with the application vary depending on the agent, the age, weight, and clinical condition of the recipient patient, and the experience and judgment of the clinician or practitioner administering the therapy, among other factors affecting the selected dosage. Dosages can range from about 0.01 mg/kg per day to about 500 mg/kg of the crystalline form of Compound 1-Na (i.e., Form A) per day. In one of the embodiments, the daily dose is preferably between about 0.01 mg/kg and 10 mg/kg of body weight.

Those of skill will readily appreciate that In one of the embodiments, the composition or formulation comprises about 0.1 mg to about 1500 mg of of the crystalline form of Compound 1-Na (i.e., Form A) per dosage form. In another embodiment, the formulation or composition comprises about 1 mg to about 100 mg of the crystalline form of Compound 1-Na (i.e., Form A). In another embodiment, the formulation comprises about 1 mg to about 50 mg. In another embodiment, the formulation comprises about 1 mg to about 30 mg. In another embodiment, the formulation comprises about 4 mg to about 26 mg. In another embodiment, the formulation comprises about 5 mg to about 25 mg. In one embodiment, the formulation comprises about 1 mg to about 5 mg. In one embodiment, the formulation comprises about 1 mg to about 2 mg.

An effective amount of a pharmaceutical agent is that which provides an objectively identifiable improvement as noted by the clinician or other qualified observer.

The pharmaceutical compositions can be included in a container, kit, pack, or dispenser together with instructions for administration.

The pharmaceutical compositions containing free form, salts, and/or solid state forms thereof of the present application (e.g., the crystalline Form A) may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active ingredient into preparations that can be used pharmaceutically. Of course, the appropriate formulation is dependent upon the route of administration chosen.

Techniques for formulation and administration of the disclosed crystalline forms or polymorphs of the application (e.g., Form A) can be found in Remington: The Science and Practice of Pharmacy, 19^{th} edition, Mack Publishing Co., Easton, Pa. (1995) or any later versions thereof.

The active ingredient can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid.

Methods for preparation of such formulations will be apparent to those skilled in the art. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

All percentages and ratios used herein, unless otherwise indicated, are by weight. Other features and advantages of the present application are apparent from the different examples. The provided examples illustrate different components and methodology useful in practicing the present application. The examples do not limit the claimed application. Based on the present disclosure the skilled artisan can identify and employ other components and methodology useful for practicing the present application.

### EXAMPLES

### Example 1: Instrument and Methodology

### X-Ray Powder Diffraction (XRPD)

### Bruker AXS C2 GADDS

X-Ray Powder Diffraction patterns were collected on a Bruker AXS C2 GADDS diffractometer using Cu Kα radiation (40 kV, 40 mA), automated XYZ stage, laser video microscope for auto-sample positioning and a HiStar 2-dimensional area detector. X-ray optics consists of a single Gobel multilayer mirror coupled with a pinhole collimator of 0.3 mm. A weekly performance check is carried out using a certified standard NIST 1976 Corundum (flat plate).

The beam divergence, i.e., the effective size of the X-ray beam on the sample, was approximately 4 mm. A 0-0 (theta-theta) continuous scan mode was employed with a sample—detector distance of 20 cm which gives an effective 20 (theta) range of 3.2°-29.7°. Typically, the sample would be exposed to the X-ray beam for 120 seconds. The software used for data collection was GADDS for XP/2000 4.1.43 and the data were analyzed and presented using Diffrac Plus EVA v15.0.0.0.

Ambient Conditions:

Samples run under ambient conditions were prepared as flat plate specimens using powder as received without grinding. Approximately 1-2 mg of the sample was lightly pressed on a glass slide to obtain a flat surface.

Non-Ambient Conditions:

Samples run under non-ambient conditions were mounted on a silicon wafer with heat-conducting compound. The sample was then heated to the appropriate temperature at 20° C./min and subsequently held isothermally for 1 minute before data collection was initiated.

### Bruker AXS D8 Advance

X-Ray Powder Diffraction patterns were collected on a Bruker D8 diffractometer using Cu Kα radiation (40 kV, 40 mA), 0-20 (theta) goniometer, and divergence of V4 and receiving slits, a Ge monochromator and a Lynxeye detector. The instrument is performance checked using a certified Corundum standard (NIST 1976). The software used for data collection was Diffrac Plus XRD Commander v2.6.1 and the data were analyzed and presented using Diffrac Plus EVA v15.0.0.0.

Samples were run under ambient conditions as flat plate specimens using powder as received. The sample was gently packed into a cavity cut into polished, zero-background (510) silicon wafer. The sample was rotated in its own plane during analysis. The details of the data collection are:
Angular range: 2 to 42° 2θ (theta)
Step size: 0.05° 2θ (theta)
Collection time: 0.5 s/step

Alternatively, sample was run under ambient conditions in transmission geometry. Approximately 10 mg of the sample was gently ground in a mortar with a pestle and tightly packed into a borosilicate glass capillary. The capillary was rotated in its own plane during analysis to minimize preferred orientation. The details of the data collection are:
Angular range: 2 to 40° 2θ (theta)
Step size: 0.0157° 2θ (theta)
Collection time: 2.7 s/step

### Nuclear Magnetic Resonance (NMR)

### ¹H NMR

NMR spectra were collected on a Bruker 400 MHz instrument equipped with an auto-sampler and controlled by a DRX400 console. Automated experiments were acquired using ICON-NMR v4.0.7 running with Topspin v1.3 using the standard Bruker loaded experiments. For non-routine spectroscopy, data were acquired through the use of Topspin alone.

Samples were prepared in DMSO-d6, unless otherwise stated. Off-line analysis was carried out using ACD Spectrus Processor 2012.

### Fourier Transform—Infra-Red (FTIR)

Data were collected on a Perkin-Elmer Spectrum One fitted with a universal Attenuated Total Reflectance (ATR) sampling accessory. The data were collected and analyzed using Spectrum v10.0.1 software.

### Differential Scanning Calorimetry (DSC)

DSC data were collected on a TA Instruments Discovery DSC equipped with a 50 position auto-sampler. The calibration for thermal capacity was carried out using sapphire and the calibration for energy and temperature was carried out using certified indium. Typically, 0.5-3 mg of each sample, in a pin-holed aluminum pan, was heated at 10° C./min from 25° C. to 180° C. A purge of dry nitrogen at 50 mL/min was maintained over the sample. The instrument control and data analysis software was TRIOS v3.2.0.3877.

### Thermo-Gravimetric Analysis (TGA)

TGA data were collected on a TA Instruments Discovery TGA, equipped with a 25 position auto-sampler. The instrument was temperature calibrated using certified alumel and nickel. Typically, 5-10 mg of each sample was loaded onto a pre-tared aluminum DSC pan and heated at 10° C./min from ambient temperature to 350° C. A nitrogen purge at 25 mL/min was maintained over the sample. The instrument control and data analysis software was TRIOS v3.2.0.3877.

### Polarized Light Microscopy (PLM)

Samples were studied on a Leica LM/DM polarized light microscope with a digital video camera for image capture. A small amount of each sample was placed on a glass slide, mounted in immersion oil and covered with a glass slip, the individual particles being separated as well as possible. The sample was viewed with appropriate magnification and partially polarized light, coupled to a λ false-color filter.

### Scanning Electron Microscopy (SEM)

Data were collected on a Phenom Pro Scanning Electron Microscope. A small quantity of sample was mounted onto an aluminum stub using conducting double-sided adhesive tape. A thin layer of gold was applied using a sputter coater (20 mA, 120 s).

### Water Determination by Karl Fischer Titration (KF)

The water content of each sample was measured on a Metrohm 874 Oven Sample Processor at 150° C. with 851 Titrano Coulometer using Hydranal Coulomat AG oven reagent and nitrogen purge. Weighed solid samples were introduced into a sealed sample vial. Approx. 10 mg of sample was used per titration and duplicate determinations were made. Data collection and analysis were carried out using Tiamo v2.2.

### Gravimetric Vapor Sorption (GVS)

SMS DVS Intrinsic:

Sorption isotherms were obtained using a SMS DVS Intrinsic moisture sorption analyzer, controlled by DVS Intrinsic Control software v1.0.1.2 (or v 1.0.1.3). The sample temperature was maintained at 25° C. by the instrument controls. The humidity was controlled by mixing streams of dry and wet nitrogen, with a total flow rate of 200 mL/min. The relative humidity was measured by a calibrated Rotronic probe (dynamic range of 1.0-100% RH), located near the sample. The weight change, (mass relaxation) of the sample as a function of % RH was constantly monitored by the microbalance (accuracy ±0.005 mg).

Typically, 5-20 mg of sample was placed in a tared mesh stainless steel basket under ambient conditions. The sample was loaded and unloaded at 40% RH and 25° C. (typical room conditions). A moisture sorption isotherm was performed as outlined below (2 scans giving 1 complete cycle). The standard isotherm was performed at 25° C. at 10% RH intervals over a 0-90% RH range. Data analysis was carried out using Microsoft Excel using DVS Analysis Suite v6.2 (or 6.1 or 6.0).

TABLE 1
Method for SMS DVS Intrinsic experiments

| Parameter | Value |
|---|---|
| Adsorption - Scan 1 | 40-90 |
| Desorption/Adsorption - Scan 2 | 90-0, 0-40 |
| Intervals (% RH) | 10 |
| Number of Scans | 2 |
| Flow rate (mL/min) | 200 |
| Temperature (° C.) | 25 |
| Stability (° C./min) | 0.2 |
| Sorption Time (hours) | 6 hour time out |

### Ion Chromatography (IC)

Data were collected on a Metrohm 761 Compact IC (for cations) using IC Net software v2.3. Accurately weighed samples were prepared as stock solutions in an appropriate dissolving solution and diluted appropriately prior to testing. Quantification was achieved by comparison with standard solutions of known concentration of the ion being analyzed.

TABLE 2
IC method for cation chromatography

| Parameter | Value |
|---|---|
| Type of method | Cation exchange |
| Column | Metrosep C 4 - 250 (4.0 × 250 mm) |
| Column Temperature | Ambient |
| (° C.) | |
| Injection (μl) | 10 |
| Detection | Conductivity detector |
| Flow Rate (mL/min) | 1.0 |
| Eluent | 1.7 mM Nitric Acid |
| | 0.7 mM Dipicolinic acid in a 5% acetone aqueous |
| | solution. |

### Example 2: Characterization of an Amorphous Form of Compound 1-Na

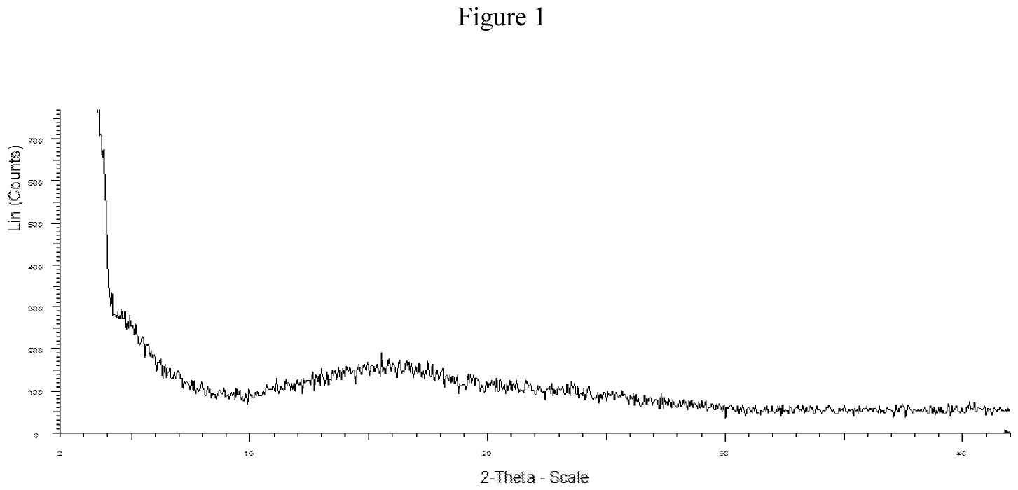

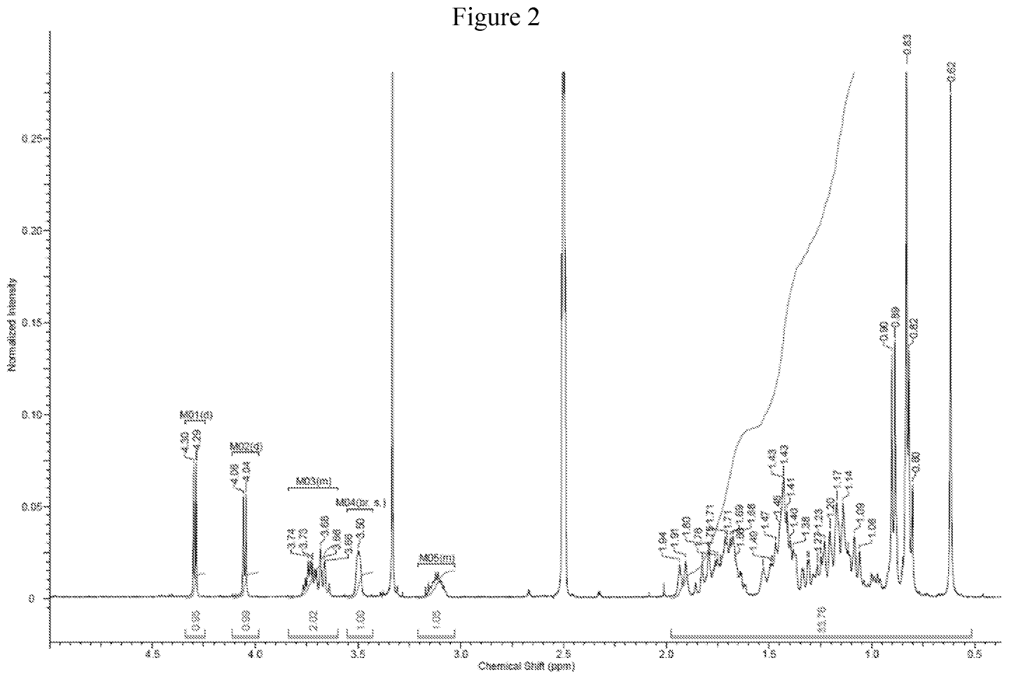

Amorphous Form of Compound 1-Na is a white powder as shown by XRPD analysis (FIG. 1). ¹H NMR spectrum was consistent with the structure of the compound (FIG. 2). Stoichiometry of the compound was determined to be 1:0.6 (API:Na). TGA analysis showed a 5.3% w/w (1.4 eq water) weight loss before decomposition. This event was related to the broad endotherm observed in the DSC thermogram at 28° C. Karl Fisher analysis showed the material to contain an average of 2.3% of water (0.63 eq). The disparity between the KF value and the weight loss observed by TGA could be due to water uptake during sample preparation. The material was shown to be deliquescent when stored at stress conditions such as 40° C./75% RH and 25° C./97% RH. Nonetheless, it was found to be physically stable for at least 1 month stored at 25° C./60% RH. The material was found to be highly soluble in water (>200 mg/mL).

TABLE 3
Characterization of Amorphous Compound 1-Na

| Data | Amorphous Compound 1-Na |
|---|---|
| XRPD | Amorphous |
| Description | White powder |
| ¹H-NMR | Consistent with structure |
| TGA | 5.3% weight loss (1.4 eq water) from RT to ca. |
| | 150° C. Decomposition observed from 150° C. |
| DSC | Broad Endotherm (onset 28.1° C., −148.7 J/g) |
| Storage @ 40° | Deliquesced in less than 3 days |
| C./75% RH | |
| Storage @ 25° | Deliquesced in less than days |
| C./97% RH | |
| Storage @ 25° | Unchanged for at least 1 month |
| C./60% RH | |
| PLM | Very small particles of irregular shape |
| KF | 2.35% |
| Aqueous Solubility | >200 mg/mL |
| (25° C.) | |

### Example 3: Crystallization of Form a of Compound 1-Na Via Suspension

An amorphous form of Compound 1-Na (204.8 mg) was suspended in acetonitrile (10.2 mL, 50 vol). The suspension was heated to 30° C. at 0.5° C./min and stirred at this temperature for 1 h. The suspension was then cooled to 0° C. at 0.2° C./min and again stirred at this temperature for 1 h. This process was repeated until 13 heating/cooling cycles were completed. Then, the sample was filtered under N₂ and dried in a vacuum oven (RT/3 mbar) for 4 hrs. The crystalline Form A of Compound 1-Na recovered: 146.7 mg. Yield=71%. Characterization data of the isolated material is summarized in Table 4.

TABLE 4
Characterization of Form A of Compound
1-Na Formed via Suspension

| Data | Form A of Compound 1-Na Formed via Suspension |
|---|---|
| Description | White powder |
| XRPD | Form A |
| ¹H-NMR | Consistent with structure |
| DSC | Endotherm (onset 27.2° C., −66.0 J/g) |
| | Endotherm (onset 159.5° C., −51.2 J/g) |
| TGA | 3.2% weight loss (0.9 eq water) from RT to ca. 150° C. |
| Storage @ 40° | Deliquesced in less than 3 days |
| C./75% RH | |
| Storage @ 25° | Deliquesced in less than 3 days |
| C./97% RH | |
| Storage @ 25° | Unchanged for at least 1 month |
| C./60% RH | |

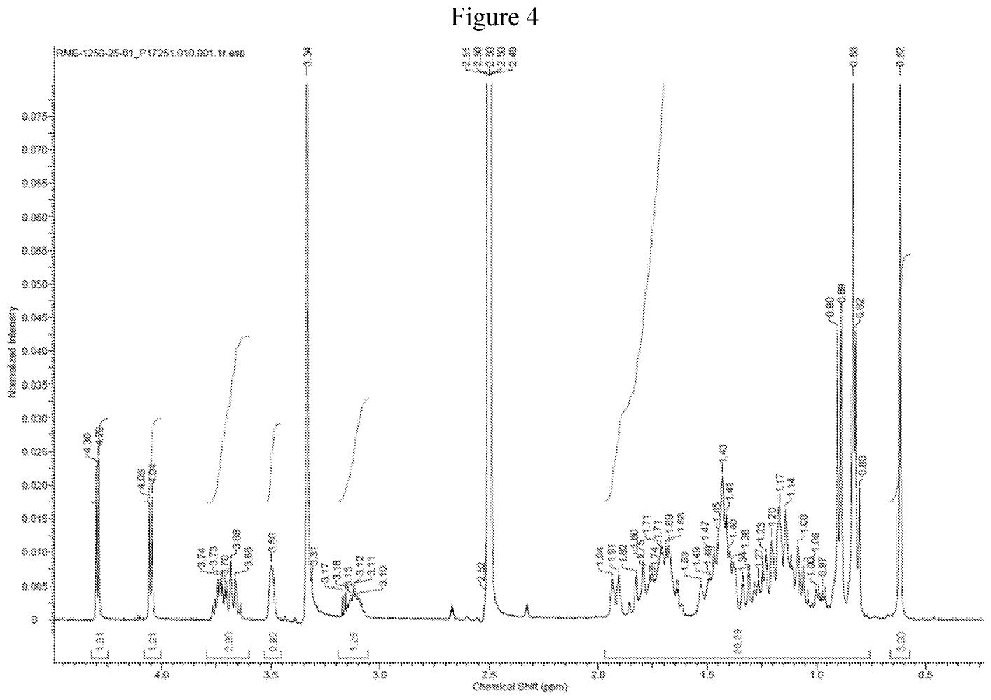

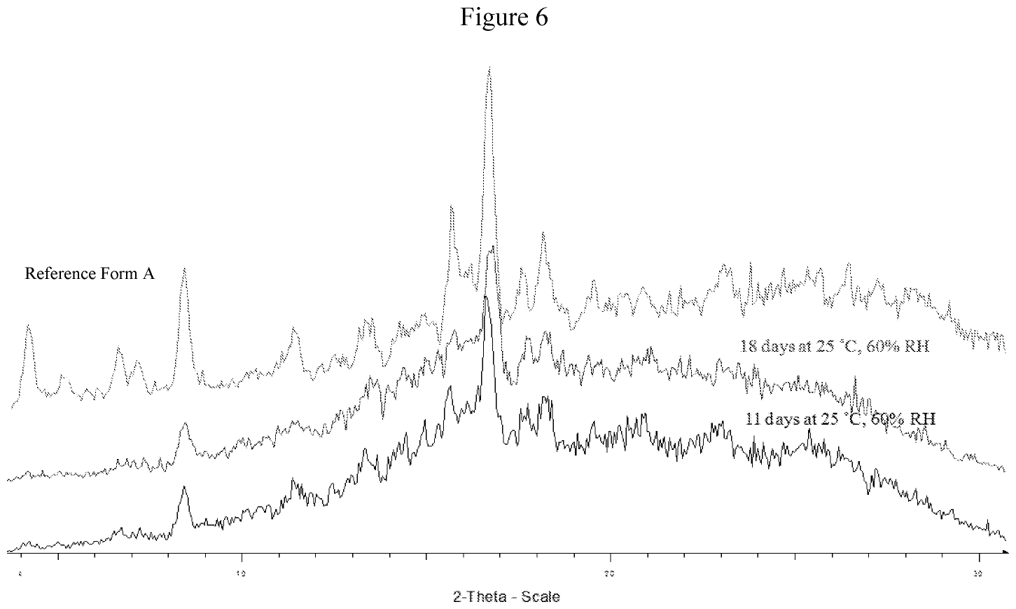

The isolated material is crystalline as shown by XRPD (FIG. 3) and no residual solvent was observed by NMR (FIG. 4). The DSC thermogram displayed a broad endothermic event consistent with the weight loss observed by TGA and another endotherm with onset at 159.5° C. consistent with a melt-decomposition (FIG. 5). The stability of the material upon storage at >60% RH conditions was successful for at least 1 month at 25° C./60% RH (FIG. 6). The material deliquesced in less than 3 days of storage at 40° C./75% RH and 25° C./97% RH.

### Example 4: Crystallization of Form a of Compound 1-Na Via Solution

An amorphous form of Compound 1-Na (20 mg) was treated with solvent aliquots (100 μL, 5 vol) (Table 5) at room temperature until dissolution was observed. Seeds of the crystalline Form A of Compound 1-Na (<1 mg, Example 3) were added to each solution. If the seeds dissolved, aliquots of the antisolvent, acetonitrile (ACN or MeCN) (20 μL, 1 vol) were added and the mixture was seeded again. The resulting solids were filtered, air-dried and analyzed by XRPD. The results are shown in Table 5.

TABLE 5
Crystallisation of Form A of Compound 1-Na via Solution

| Sample | | | ACN | | |
|---|---|---|---|---|---|
| No. | Solvent | Vol | (μL) | Comments | XRPD |
| 1 | Acetone | 10 | 0 | Crystallized occurred | Form A |
| | | | | just after seeding | |
| 2 | EtOH | 5 | 500 | Particles observed when | Form A |
| | | | | MeCN = 150 μl. | |
| | | | | (EtOH:MeCN, 40:60) | |
| 3 | Water | 5 | 200 | Seeds dissolved quickly | No |
| | | | | | crystals |
| | | | | | formed |
| 4 | THF | 5 | 200 | A powder + gummy | Form A |
| | | | | material observed | |

Form A of Compound 1-Na was obtained from all the non-aqueous solvents tested. In the case of acetone, crystallization started as soon as seeds were added to the solution without the need for the addition of antisolvent. In the case of ethanol, a turbid solution was observed at 60% ratio of acetonitrile and the seeds remained in suspension. Addition of antisolvent resulted in a large amount of solid.

### Example 5: Scale-Up Crystallization of Form a of Compound 1-Na Via Solution at 30° C.

An amorphous form of Compound 1-Na (0.5 g) was dissolved in ethanol:acetonitrile 30:70 (6 mL, 12 vol) at 30° C. The solution was rapidly cooled to 20° C. and seeds of Form A of Compound 1-Na (Example 3, 2.5 mg, 0.5% w/w) were added. Acetonitrile (7.2 mL, 14.4 vol, to give 15% EtOH (v/v)) was added dropwise at 20° C. After addition the sample was cooled to 5° C. at 0.1° C./min and was stirred at this temperature for 16 hrs. The solid was isolated under vacuum filtration, air-dried for 20 min (analyzed by XRPD) and placed in the vacuum oven (RT/3 mbar) for 3 days. The crystalline Form A of Compound 1-Na was recovered in the amount of 0.325 g (yield=65%).

### Example 6: Scale-Up Crystallization of Form a of Compound 1-Na Via Solution at 20° C.

An amorphous form of Compound 1-Na (0.5 g) was dissolved in dried ethanol:acetonitrile 30:70 (6 mL, 12 vol) at 20° C. Seeds of Form A of Compound 1-Na (Example 3, 2.5 mg, 0.5% w/w) were added. Acetonitrile (7.2 mL, 14.4 vol, to give 15% EtOH) was added dropwise at this temperature. The sample was cooled to 5° C. at 0.1° C./min and was stirred at this temperature for 16 hrs. The solid was isolated under vacuum filtration, air-dried for 20 min (analyzed by XRPD) and placed in the vacuum oven (RT/3 mbar) for 3 days. The crystalline Form A of Compound 1-Na was recovered in the amount of 0.270 g (yield=54%).

### Example 7: Results of Scale-Up Crystallization of Form a of Compound 1-Na Via Solution

The isolated materials from Examples 5 and 6 were characterized and the results are summarized in Table 6.

TABLE 6
Characterization of Form A of Compound 1-Na

| Method | Example 5 (via solution at 30° C.) | Example 6 (via solution at 20° C.) |
|---|---|---|
| XRPD | Form 1 (Na Salt) | Form 1 (Na Salt) |
| ¹H-NMR | Consistent with structure | n/d |
| Sample dried at RT for 3 | MeCN (0.4% w/w, 0.05 eq), EtOH | |
| days | (0.6% w/w, 0.06 eq) | |
| ¹H-NMR | Consistent with structure | n/d |
| Sample dried at 40° C. for 3 | MeCN (0.06% w/w, 0.01 eq) | |
| days | | |
| TGA | 2.3% weight loss (0.6 eq water) | 2.7% weight loss (0.7 eq water) |
| | from RT to ca. 150° C. | from RT to ca. 150° C. |
| DSC | Endotherm (onset 28.8° C., −74.9 | Endotherm (onset 29.2° C., −61.6 |
| | J/g) | J/g) |
| | Endotherm (onset 165.3° C., −30.4 | Endotherm (onset 169.4° C., −29.1 |
| | J/g) | J/g) |
| Storage @ 25° C./60% RH | Unchanged for at least 1 month | n/d |
| Storage @ 25° C./97% RH | Deliquesced in three days | |
| Storage @ 40° C./75% RH | Deliquesced in three days | |
| VT-XRPD | Unchanged | n/d |
| GVS | 4% w/w of water (1.1 eq) uptake | n/d |
| | from 40% to 80% RH | |
| | 24% w/w (6.6 eq) from 80% to 90 | |
| | % RH. | |
| | Sample deliquesced during the | |
| | analysis | |
| PLM | Small particles along with | n/d |
| | irregular shape particles >300 μm | |
| SEM | Small acicular particles along with | |
| | large lath particles | |
| KF | 2.6% | n/d |
| Aqueous Solubility (25° C.) | >200 mg/mL | n/d |

n/d: not determined

XRPD analysis of the samples showed that both solids were Form A (FIG. 7). Thermal analyses of the materials showed the same thermal profile. The material isolated in Example 5 (FIG. 8) and Example 6 (FIG. 9), displayed a weight loss from room temperature to ca. 150° C. of 2.3% w/w and 2.7% w/w, respectively, as measured by TGA (2.5% w/w average). DSC thermograms of both samples showed a broad endothermic event observed with onset at ca. 29° C. Melt-decomposition of the compound was determined at 165.3° C. and 169.4° C. (167° C. average). The small differences between the data from the thermograms of Examples 5 and 6 were assumed to be related to the manual integration. To this point, both materials were consistent with each other, and further characterization was only performed on isolated material from Example 5.

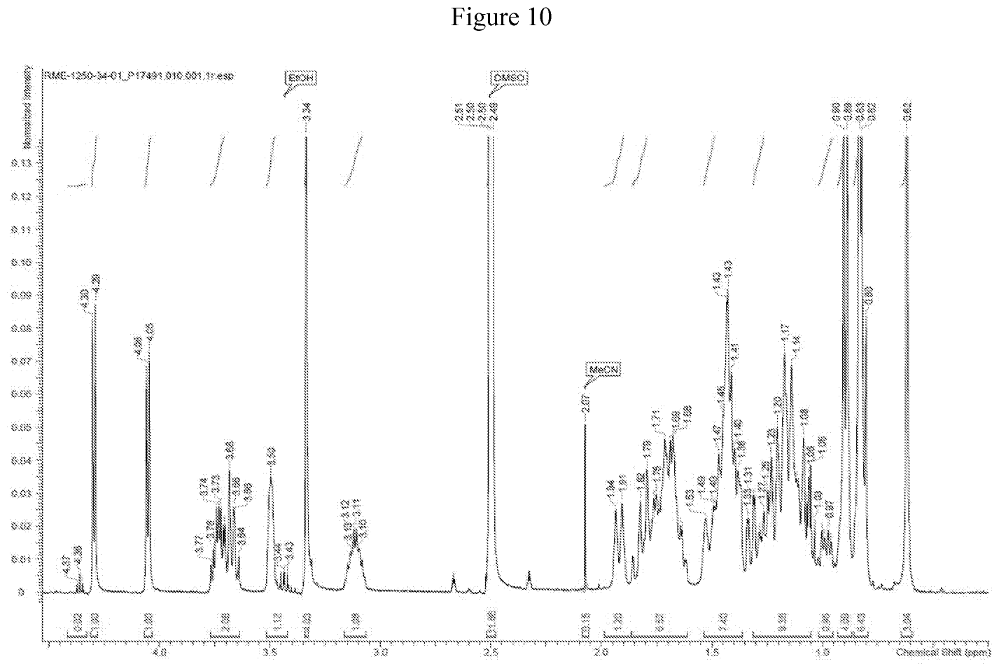

The crystalline Form A of Compound 1-Na (Example 5) displayed small amounts of residual acetonitrile (0.4% w/w, 0.5 eq) and ethanol (0.6% w/w, 0.6 eq) by NMR (FIG. 10). Consistent with the TGA data, Karl Fisher analysis determined 2.6% (0.7 eq) of water in the sample. Therefore, the events observed in the TGA and DSC corresponded mainly to the loss of water (0.7 eq).

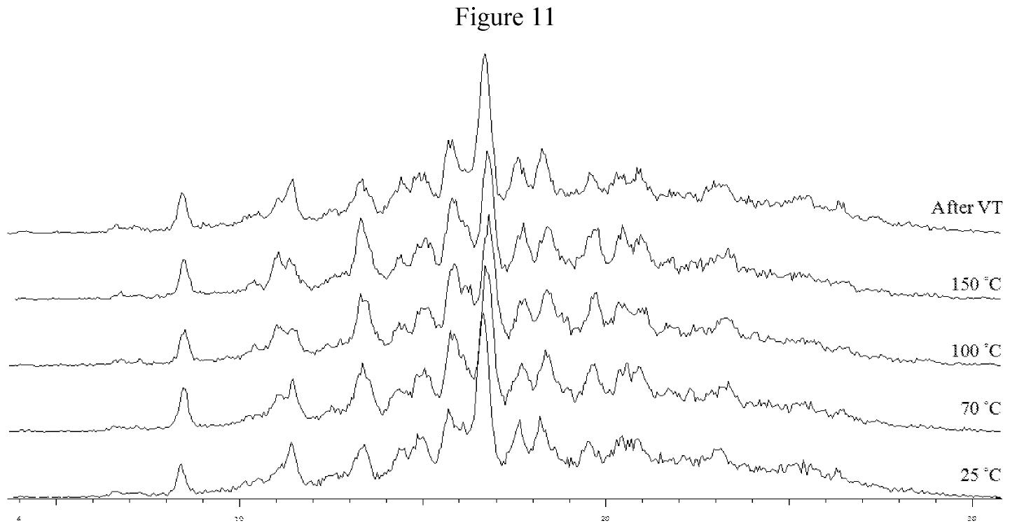

VT-XRPD analysis showed the material to be unchanged upon heating and therefore the material was not a hydrate (FIG. 11).

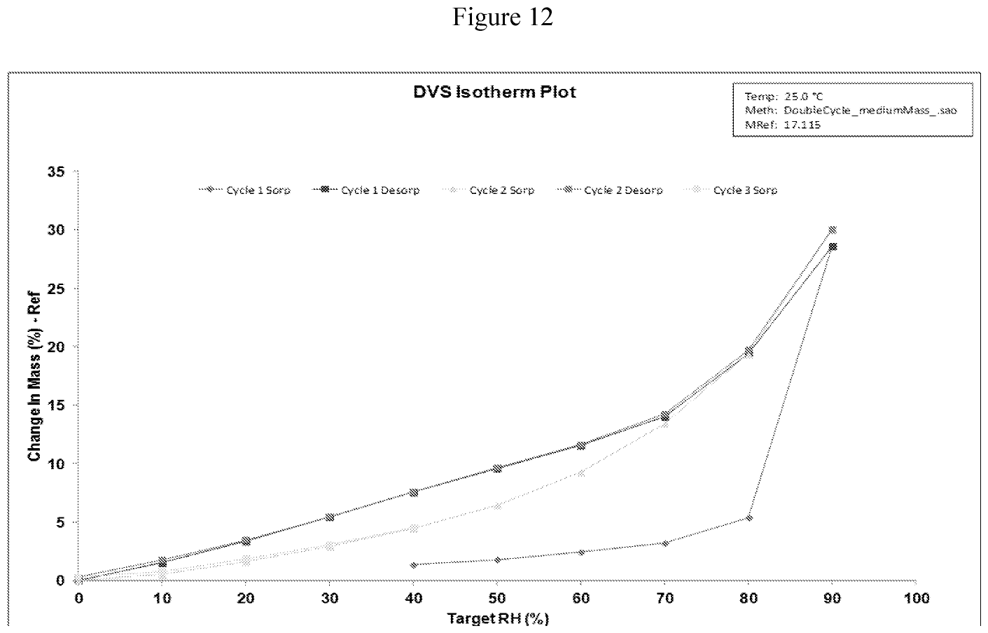

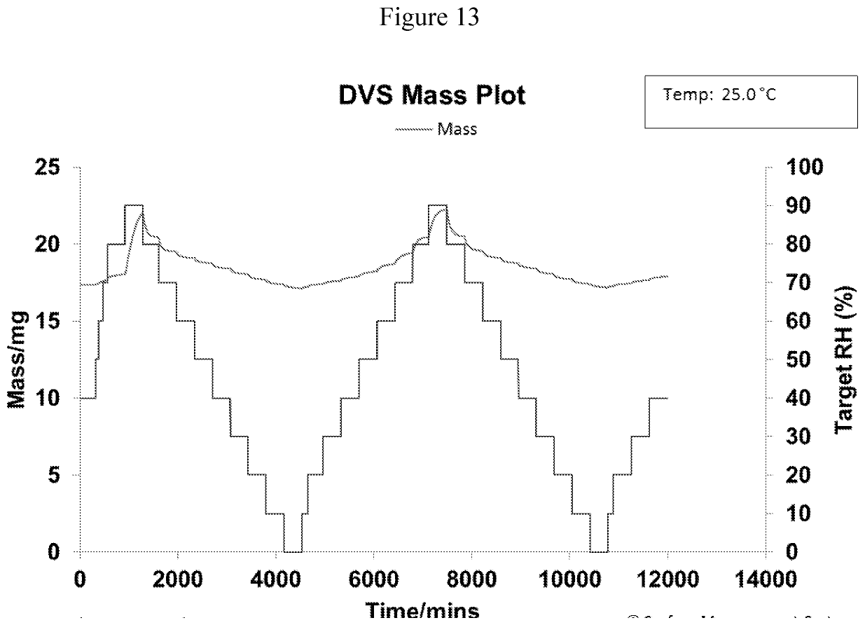

The hygroscopicity of the material was investigated by GVS. The material showed a moderate hygroscopic profile from 40% to 80% RH with a water uptake of 4% w/w (1.1 eq) and a very hygroscopic profile at RH greater than 80% (24% water (6.6 eq) uptake). During this analysis, the sample deliquesced and no further analysis could be performed after the GVS. Stability testing of the material under the stress conditions of 40° C./75% RH and 25° C./97% RH resulted in a deliquesced sample after 3 days of storage at those conditions. However, the material was found to be stable for at least one month when stored at 25° C./60% RH (FIGS. 12 and 13).

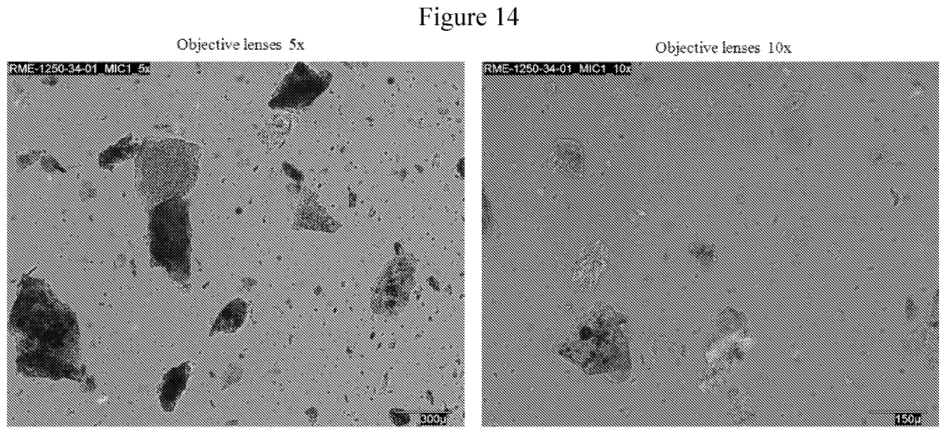

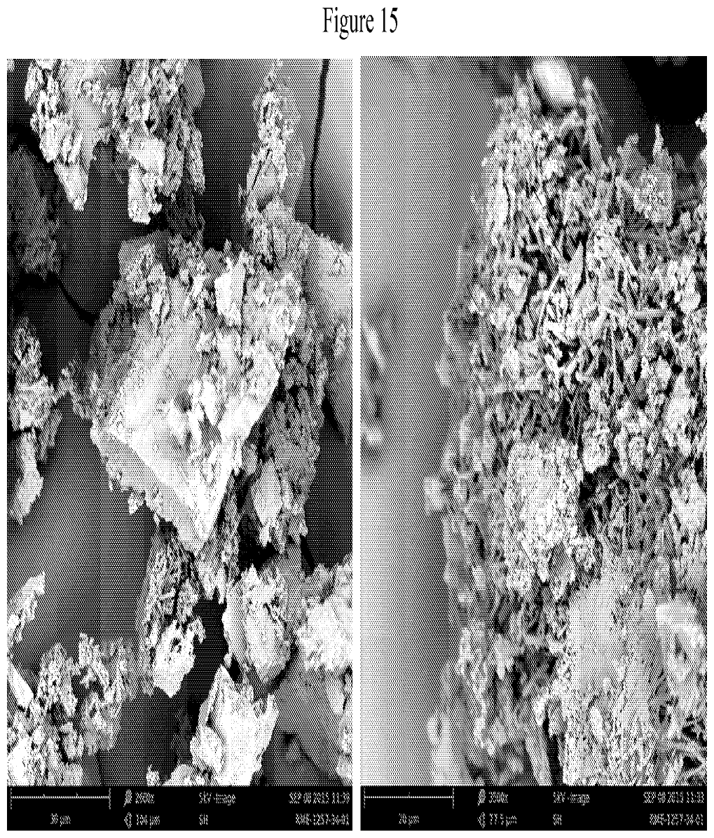

Observation of the material under the optical microscope showed solids composed of small particles. Images from the electronic microscope (SEM) showed the material to be composed of small acicular particles along with large lath and irregular shape particles (FIGS. 14 and 15).

The aqueous solubility of the material at 25° C. was determined as greater than 200 mg/mL.

IC analysis of the material determined the same result as for the amorphous material (0.6 eq of sodium).

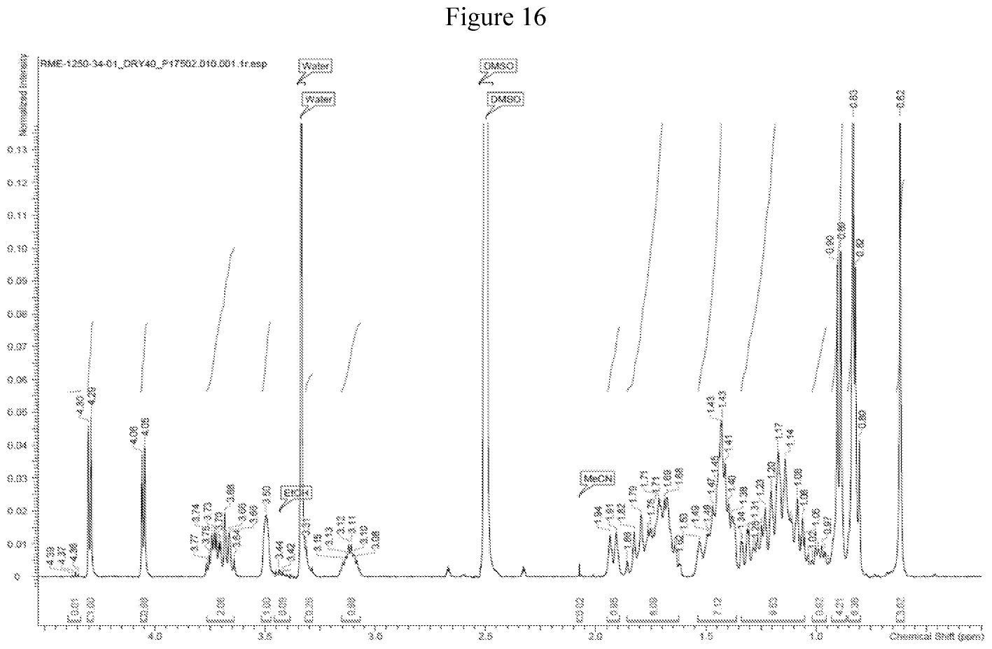

The amount of residual solvent in the aliquot dried at 40° C. was reduced to trace amounts when compared with the sample dried at room temperature. Residual acetonitrile (0.06% w/w) and ethanol were detected by NMR (FIG. 16). Also, XRPD analysis of this aliquot showed the material to be unchanged after drying (FIG. 17).

### Example 8: High Resolution XRPD Experiments and Analysis

### Data Collection

High Resolution XRPD was collected on a Bruker D8 diffractometer using Cu Kα radiation (40 kV, 40 mA), 0-20 (theta) goniometer, and divergence of V4 and receiving slits, a Ge monochromator and a Lynxeye detector. The instrument is performance checked using a certified Corundum standard (NIST 1976). The software used for data collection was Diffrac Plus XRD Commander v2.6.1 and the data were analyzed and presented using Diffrac Plus EVA v15.0.0.0.

Sample was run under ambient conditions in transmission geometry. Approximately 10 mg of the sample was gently ground in a mortar with a pestle and tightly packed into a borosilicate glass capillary. The capillary was rotated in its own plane during analysis to minimize preferred orientation. The details of the data collection are:
Angular range: 2 to 40° 2θ (theta)
Step size: 0.0157° 2θ (theta)
Collection time: 2.7 s/step

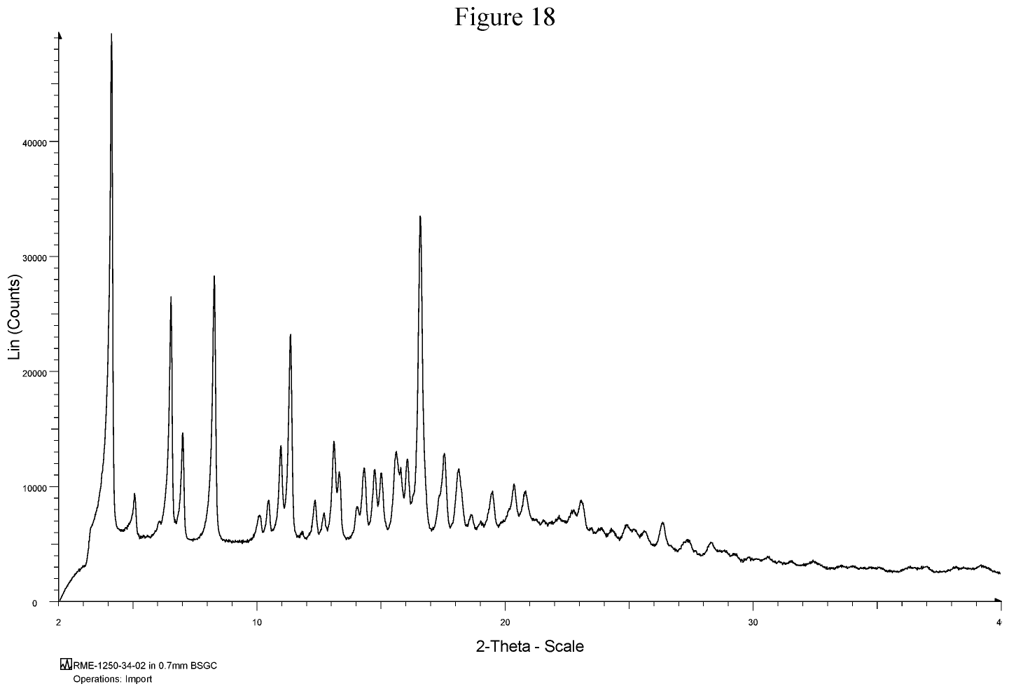

The XRPD pattern is shown in FIG. 18. Indexing of the XRPD results by three different indexing programs returned similar results, with the total volume of the basic unit cell being around 8000-8300 Å³, indicating an orthorhombic crystal system with the following unit cell parameters:

a=8.7 Å, b=27.0 Å and c=34.8 Å (volume 8181.4 Å³).

### Example 9: Scale-Up Crystallization of Form a of Compound 1-Na at 11 g Scale

The experimental set up of this experiment involved an automated reactor system that prevents condensation when working at low temperatures. In addition, the experiment was performed in dried solvents and under a positive pressure of nitrogen.

An amorphous form of Compound 1-Na (11.5 g) was mixed with dried ethanol:acetonitrile (30:70) (134 ml, 11.6 vol). The mixture was heated from 14° C. to 30° C. at 1° C./min and stirred at this temperature for 5 min. The sample was cooled to 20° C. at 5° C./min. At 20° C., seeds of Form A of Compound 1-Na (58.89 mg, 0.5% w/w) were added. Acetonitrile (158 ml, 13.7 vol) was added over 55 min. Precipitation was observed after the addition of 126 ml of acetonitrile (10.9 vol) ca. 14% ethanol. The sample was cooled to 5° C. at 0.1° C./min and was stirred at this temperature overnight. The solid was isolated by vacuum filtration under a nitrogen flow. The filter cake was washed with acetonitrile (15 ml, 1.3 vol) and the material was scratched from the wall of the vessel. The solid was dried in a vacuum oven (40° C./3 mbar) overnight. The crystalline Form A of Compound 1-Na was recovered in the amount of 7.9 g (yield=69%). Characterization and a summary of the results are shows in Table 7.

TABLE 7
Characterization Data of 11 g Scale-up Crystallization
of Form A of Compound 1-Na

| Sample ID | |
|---|---|
| Data | Scaled-up Crystalline Form A of Compound 1-Na |
| Description | white powder |
| XRPD | INT-767 Form 1 + amorphous background |
| ¹H NMR | Consistent with structure. |
| | EtOH (1.2% w/w, 0.13 eq) |
| | MeCN (0.86% w/w, 0.10 eq) |
| TGA | 3.2% weight loss from RT to ca. 150° C. |
| DSC | Endotherm (onset 28.8° C., −48.5 J/g) |
| | Endotherm (onset 150.4° C., −21.7 J/g) |
| 12 days Storage @ | Unchanged |
| 25° C./60% RH | |
| 12 days Storage @ | Unchanged |
| 40° C./60% RH | |
| GVS | 2.6% w/w of water uptake from 40% to 60% RH. |
| | Total of 8% w/w water uptake. |
| | The process is reversible with a hysteresis of ca. |
| | 1% w/w |
| XRPD post GVS | Unchanged |
| ¹H NMR | Consistent with structure. No residual solvents |
| post GVS | detected |
| PLM | Very small particles |
| SEM | Small acicular particles that tend to agglomerate |
| KF | Replicate A = 2.4% w/w |
| | Replicate B = 2.7% w/w |

### Example 10: Crystallization of Form a of Compound 1-Na Via Mobile Slurry

The amorphous form of Compound 1-Na (504.4 mg) was mixed with dried ethanol:acetonitrile (30:70) (6 ml, 12 vol). The sample was initially heated to 30° C. and stirred at this temperature for 15 min. Then, it was heated to 35° C. and stirred at this temperature for 5 min. The sample was rapidly cooled to 20° C. and seeds of crystalline Form A of Compound 1-Na (4 mg, 0.8% w/w) were added. Acetonitrile (7.2 ml, 14.4 vol) was added dropwise. Precipitation was observed during the addition. The sample was then cooled to 5° C. at 0.1° C./min and stirred at this temperature overnight. After this time a mobile slurry was observed. The solid was isolated by vacuum filtration, washed with acetonitrile and dried under a nitrogen flow for 1 h. The solid was dried in a vacuum oven (40° C./3 mbar) overnight. The crystalline Form A of Compound 1-Na was recovered in the amount of 196.4 mg (yield=39%). The isolated material was found to be crystalline and consistent with crystalline Form A of Compound 1-Na.

Without being bound by any theory, observation of mobile slurry after the addition of the antisolvent could be due to the higher purity and lower water content of this batch.

### Example 11: Crystallization of the Crystalline Form a of Compound 1-Na from Ethanol/Acetonitrile at 20° C.

The amorphous form of Compound 1-Na (0.5 g) was dissolved in dried ethanol:acetonitrile 30:70 (6 ml, 12 vol) at 20° C. Then, seeds of crystalline Form A of Compound 1-Na (2.5 mg, 0.5% w/w) were added. Acetonitrile (7.2 ml, 14.4 vol, to give 15% EtOH) was added dropwise at this temperature. The sample was cooled to 5° C. at 0.1° C./min and was stirred at this temperature for 16 hrs. The solid was isolated under under vacuum filtration, air-dried for 20 min (analysed by XRPD) and placed in the vacuum oven (RT/3 mbar) for 3 days. The crystalline Form A of Compound 1-Na was recovered in the amount of 0.270 g (yield=54%). Corresponding XRPD diffractogram is shown in FIG. 19 and list of peaks and their intensities is shown in Table 8.

TABLE 8
List of Peaks versus Intensities (Peak Position
Accuracy = ±0.2 °2θ)

| Angle 2-Theta ° | Intensity % |
|---|---|
| 4.1 | 59.6 |
| 5.1 | 14.3 |
| 6.6 | 30.4 |
| 7.0 | 17.4 |
| 8.3 | 64.6 |
| 10.1 | 10.6 |
| 10.5 | 8.0 |
| 11.0 | 15.8 |
| 11.4 | 30.2 |
| 12.3 | 8.8 |
| 13.2 | 22.1 |
| 13.4 | 18.9 |
| 14.1 | 10.3 |
| 14.4 | 19.9 |
| 14.8 | 18.9 |
| 15.1 | 14.5 |
| 12.7 | 8.3 |
| 15.7 | 47.8 |
| 16.1 | 32.1 |
| 16.6 | 100 |
| 17.6 | 25.4 |
| 18.2 | 37.5 |
| 18.7 | 13.1 |
| 19.5 | 16.3 |
| 20.9 | 16.8 |
| 23.1 | 18.4 |

### Example 12: Crystallization of Compound 1-OH

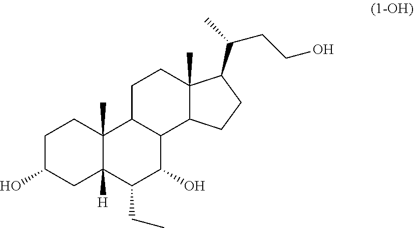 [SMILES: CC[C@H]1[C@@H](O)C2C3CC[C@H]([C@H](C)CCO)[C@@]3(C)CCC2[C@@]2(C)CC[C@@H](O)C[C@@H]12]

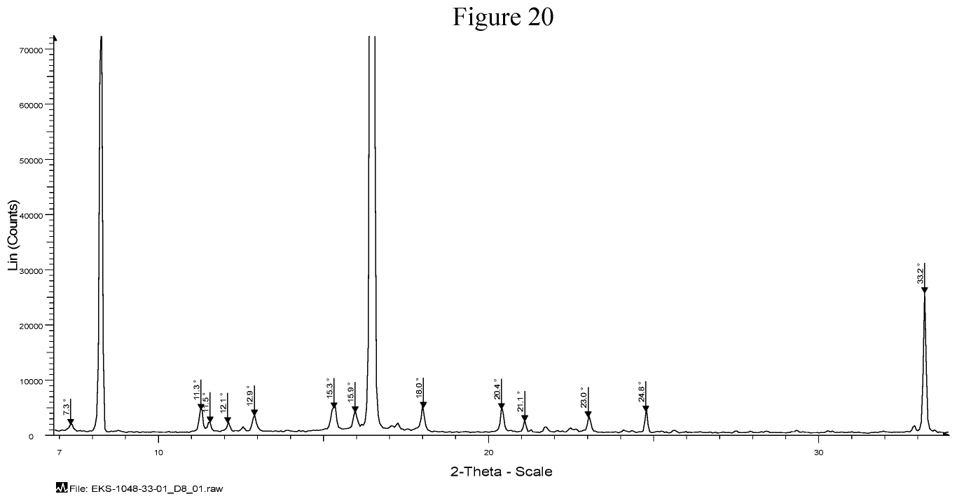

A sample of crystalline form of Compound 1-OH was prepared by slow evaporation from a solution in DCM. The crystal exhibited plate morphology of approximate dimensions 0.35×0.30×0.10 mm for the analysis. XRPD of the Crystalline Form of Compound 1-OH is shown in FIG. 20. List of peaks and intensities is shown in Table 9.

TABLE 9
List of Peaks versus Intensities (Peak Position
Accuracy = ±0.2 °2θ)

| Angle 2-Theta ° | Intensity % |
|---|---|
| 7.3 | 0.3 |
| 8.2 | 12.8 |
| 11.3 | 0.8 |
| 11.5 | 0.4 |
| 12.1 | 0.4 |
| 12.9 | 0.6 |
| 15.3 | 0.8 |
| 15.9 | 0.7 |
| 16.4 | 100 |
| 18.0 | 0.8 |
| 20.4 | 0.8 |
| 21.1 | 0.4 |
| 23.0 | 0.6 |
| 24.8 | 0.7 |
| 33.2 | 4.5 |

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

1. A crystalline form of Compound 1-Na, characterized by having X-ray powder diffraction (XRPD) peaks at approximately 8.5, 15.8, and 16.7° 2θ (theta) using Cu Kα radiation.

2. The crystalline form of claim 1, characterized by having XRPD peaks at approximately 4.0, 8.5, 15.8, 16.7, 17.8, and 18.2° 2θ (theta) using Cu Kα radiation.

3. The crystalline form of claim 1, characterized by having XRPD peaks at approximately 4.0, 6.6, 7.1, 8.5, 11.5, 13.5, 15.8, 16.7, 17.8, and 18.2° 2θ (theta) using Cu Kα radiation.

4. The crystalline form of claim 1, characterized by having an XRPD pattern substantially similar to that shown in FIG. 3, FIG. 7, FIG. 17 or FIG. 19.

5. The crystalline form of claim 1, further characterized by a Differential Scanning calorimetry (DSC) having an onset temperature between about 165° C. and about 169° C.

6. The crystalline form of claim 1, further characterized by a DSC having an onset temperature at approximately 165° C. or 167° C.

7. The crystalline form of claim 1, further characterized by a DSC having an onset temperature at about 30° C.

8. The crystalline form of claim 1, further characterized by a DSC having an onset temperature at approximately 29° C.

9. The crystalline form of claim 1, further characterized by a DSC having a first onset temperature at about 30° C. and a second onset temperature between about 165° C. and about 169° C.

10. The crystalline form of claim 1, further characterized by a DSC having a first onset temperature at approximately 29° C. and a second onset temperature at approximately 165° C. or 169° C.

11. A crystalline form of Compound 1-Na, characterized by having an orthorhombic crystal system with the following unit cell parameters: a=approximately 8.7 Å, b=approximately 27.0 Å, and c=approximately 34.8 Å.

12. A pharmaceutical composition comprising the crystalline form of any one of claims 1-11, and a pharmaceutically acceptable diluent, excipient or carrier.

13. A method of treating or preventing an FXR-medated disease or disorder in a subject in need thereof, comprising administering a therapeutically effective amount of the crystalline form of any one of claims 1-11.

14. A method of modulating FXR in a subject in need thereof, comprising administering a therapeutically effective amount of the crystalline form of any one of claims 1-11.

15. A method of preparing a crystalline form of Compound 1-Na, comprising:
(a) dissolving amorphous Compound 1-Na in a solvent to form a solution;
(b) cooling the solution;
(c) repeating step (a) and step (b) for one or more times; and
(f) filtering the product from step (c) and drying the product under vacuum.

16. A method of preparing a crystalline form of Compound 1-Na, comprising:
(a) dissolving amorphous Compound 1-Na in a solvent to form a solution;
(b) optionally cooling the solution comprising Compound 1-Na;
(c) adding a crystalline seed of the crystalline Form A of Compound 1-Na to the solution;
(d) adding acetonitrile to the solution;
(e) cooling the solution; and
(f) isolating the crystalline Form A of Compound 1-Na under vacuum filtration.

17. A crystalline form of claim 1, characterized by having X-ray powder diffraction (XRPD) peaks at 8.5±0.2° two theta, 15.8±0.2° two theta, and 16.7±0.2° two theta using Cu Kα radiation.

18. The crystalline form of claim 1, characterized by having XRPD peaks at 4.0±0.2° two theta, 8.5±0.2° two theta, 15.8±0.2° two theta, 16.7±0.2° two theta, 17.8±0.2° two theta, and 18.2±0.2° two theta using Cu Kα radiation.

19. The crystalline form of claim 1, characterized by having XRPD peaks at 4.0±0.2° two theta, 6.6±0.2° two theta, 7.1±0.2° two theta, 8.5±0.2° two theta, 11.5±0.2° two theta, 13.5±0.2° two theta, 15.8±0.2° two theta, 16.7±0.2° two theta, 17.8±0.2° two theta, and 18.2±0.2° two theta using Cu Kα radiation.

20. A crystalline form of claim 11 characterized by having an orthorhombic space group P2₁2₁2₁.

21. A crystalline form of claim 1 characterized by having an orthorhombic space group P2₁2₁2₁.
